SEQUENCE LISTING (39 sequences)

SEQ ID NO: 1 - DNA, 28 bp, Artificial Sequence
  Mouse Scotin-specific primer
gctgtatagagggccacatgtgttcact

SEQ ID NO: 2 - DNA, 28 bp, Artificial Sequence
  Mouse scotin-specific antisense primer
aaagacagtgcagggagaaaccagagtg

SEQ ID NO: 3 - DNA, 20 bp, Artificial Sequence
  Mouse GADPH-specific sense primer
tggactgtggtcatgagccc

SEQ ID NO: 4 - DNA, 19 bp, Artificial Sequence
  Mouse GADPH-specific antisense primer
cagcaatgcatcctgcacc

SEQ ID NO: 5 - DNA, 21 bp, Artificial Sequence
  Primer for 3′ end of mouse Scotin mRNA
cccgggaaggacagtgacatc

SEQ ID NO: 6 - DNA, 22 bp, Artificial Sequence
  Primer 2 for 3′ end of mouse Scotin mRNA
ttcaagtgaggaagaaaacagg

SEQ ID NO: 7 - DNA, 20 bp, Artificial Sequence
  Primer 7
gggcctgcacagctcaccat

SEQ ID NO: 8 - DNA, 18 bp, Artificial Sequence
  Primer 8
cggccggggcggggcaag

SEQ ID NO: 9 - DNA, 22 bp, Artificial Sequence
  Primer 9
tcagggaattgtctttagggaa

SEQ ID NO: 10 - DNA, 33 bp, Artificial Sequence
  Primer 10
tatgtcagggttcggagcgaccgtcgccattgg

SEQ ID NO: 11 - DNA, 53 bp, Artificial Sequence
  Primer 11
cgcgctcgagctacttgtcatcgtcgtccttgtaatcgggaattgtctttagg

SEQ ID NO: 12 - DNA, 28 bp, Artificial Sequence
  Primer 12
acgacgttgtaaaacgacggccagagaa

SEQ ID NO: 13 - DNA, 20 bp, Artificial Sequence
  Primer 13
aggccgcgggcgcagccatg

SEQ ID NO: 14 - DNA, 20 bp, Artificial Sequence
  Primer 14
cagaccgcggggatcgaatt

SEQ ID NO: 15 - DNA, 33 bp, Artificial Sequence; identical to SEQ ID NO: 10
  Primer 15

SEQ ID NO: 16 - DNA, 47 bp, Artificial Sequence
  Primer 16
cgcgctcgagctacttgtcatcgtcgtccttgtaatccagacagcag

SEQ ID NO: 17 - DNA, 25 bp, Artificial Sequence
  Primer 17
cttcgccgttggcctgaccatcttt

SEQ ID NO: 18 - DNA, 25 bp, Artificial Sequence
  Primer 18
ccacacttggaggctgaggataagg

SEQ ID NO: 19 - DNA, 25 bp, Artificial Sequence
  Primer 19
gccctcgagcctccgggtgcccatg

SEQ ID NO: 20 - DNA, 27 bp, Artificial Sequence
  Primer 20
gcggaattcgcgggggtggaaaatctg

SEQ ID NO: 21 - protein, 19 aa, Mus musculus
PYHESLAGASQPPYNPTYK

SEQ ID NO: 22 - protein, 20 aa, Homo sapiens
YHETLAGGAAAPYPASQPPK

SEQ ID NO: 23 - DNA, 1845 bp, Mus musculus
cggccggggcggggcaaggaggctagggccgcgctggtcgcggaggttgcggcggcaccgtggtcttgggcttggtccgtctgttcgtccgtccgttggtctgtcccgccatggctgcgccggcgccctctctgtggaccctattgctgctgctgttgctgctgccgccgcctccgggtgcccatggtgagctgtgcaggccctttggtgaagacaattcgatcccagtgttctgtcctgatttctgttgtggttcctgttccaaccaatactgctgctcggacgtgctgaggaaaatccagtggaatgaggaaatgtgtcctgagccagagtccagattttccacccccgcggaggagacacccgaacatctgggttcagcgctgaaatttcgatccagttttgacagtgaccctatgtcagggttcggagcgaccgtcgccattggcgtgaccatctttgtggtgtttattgccactatcatcatctgcttcacctgctcctgctgctgtctgtataagatgtgctgcccccaacgccctgtcgtgaccaacaccacaactactaccgtggttcatgccccttaccctcagcctcaacctcaacctgtggcccccagctatcctggaccaacataccagggctaccatcccatgcccccccagccaggaatgccagcagcaccctacccaacgcagtacccaccaccctacctggcccagcccacagggccgccaccctaccatgagtccttggctggagccagccagcctccatacaacccgacctacatggattccctaaagacaattccctgaacctgcccccagcctctttggtgccatttatgtcgtgtgtgagtgagtgatacgcagagttctttactgctgtctgtggtgtgtgtgccttgtctagacatgtggcttcctctgctgttgaccaggtaggcgcaagtcttaccagtgtgggtcgggaccaacctgttttcttcctcacttgaaattgtactttctgaaatttcaagcaaattaaaaacaataaggtaggaggtatttcccacgtcaccccaaggtgaccagccatggcctgtcatacttaggagagcaagctttttgcgggtacagagcaggctttggggggtaaccagctagctgctgctaggcctttattcccagggtttggctgcattggcagtgaggcaggtggctgggggtgacaccaggtgacaaggggactcagtggcagggggtcacaccaggcagaacaccatacactctccatcagctgtctgtctggatgtcactgtccttcccggggctgtatagagggccacatgtgttcactattcaggctccactgggggaattttcctacctttgctggcttggctcctgctcccaggccagggacctcggtctgtctactacacactctggtttctccctgcactgtctttttactgttagccaaacattttgcctgttttctgtctccagatgtgtgataattggtgtgaggttgaaatccctggttcctggaggacagacaacctgacctccgactgtcagtttcccttgacaccatcttcatagaaatacctgactcctgtaccacagtccagtttgtcccagtagcagggacaccaaggccaatgggttatctggaccaaaggtggggtggagggcctaggtggtatctccggcccagatgtgaatacctccatattccctgttggttcctgtttcactggctgttttagctttgtgttgattggtgtttctgagcattcagactccgcaccctcatttctaataaatgcaacattggacccgcaaaaaaaaaaaa

SEQ ID NO: 24 - DNA, 2166 bp, Homo sapiens
cggacagaggttccgggaaccagccgggccggggcggggcggggcgagggagaggggcggccgcgcggatcactgaggctgtggcggcactgcgcccggcgctcgcgtccgtccgcccgtccgcccgcccagccatgactgcgccggtccccgcgccgcggatcctgttgccgttgctgttgctgctgctgctaacgccgcctccgggtgcacgtggtgaggtgtgtatggcttcccgtggactcagcctcttccccgagtcctgtccagatttctgctgtggtacctgtgatgaccaatactgctgctctgacgtgctgaagaaatttgtgtggagcgaggaaaggtgtgctgtgcctgaggccagcgtgcctgccagtgtagagccggtggagcagctgggctcggcgctgaggtttcgccctggctacaacgaccccatgtcagggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 25 - protein, 235 aa, Mus musculus
MAAPAPSLWTLLLLLLLLPPPPGAHGELCRPFGEDNSIPVFCPDFCCGSCSNQYCCSDVLRKIQWNEEMCPEPESRFSTPAEETPEHLGSALKFRSSFDSDPMSGFGATVAIGVTIFVVFIATIIICFTCSCCCLYKMCCPQRPVVTNTTTTTVVHAPYPQPQPQPVAPSYPGPTYQGYHPMPPQPGMPAAPYPTQYPPPYLAQPTGPPPYHESLAGASQPPYNPTYMDSLKTIP

SEQ ID NO: 26 - protein, 240 aa, Homo sapiens
MTAPVPAPRILLPLLLLLLLTPPPGARGEVCMASRGLSLFPESCPDFCCGTCDDQYCCSDVLKKFVWSEERCAVPEASVPASVEPVEQLGSALRFRPGYNDPMSGFGATLAVGLTIFVLSVVTIIICFTCSCCCLYKTCRRPRPVVTTTTSTTVVHAPYPQPPSVPPSYPGPSYQGYHTMPPQPGMPAAPYPMQYPPPYPAQPMGPPAYHETLAGGAAAPYPASQPPYNPAYMDAPKAAL

SEQ ID NO: 27 - DNA, 2130 bp, Homo sapiens
tctagctcagtcctggcccactgcgccagcgctgagcctgccagggctggggctggggatcaccttgggatgatggtgtcagtcccagggggcaggagatcgagtgtcctctgagctggcgactgggcctgtagaagggaaccggcatttgtggagtgtccactgagtgccaagcacgtggtgaggtgtgtatggcttcccgtggactcagcctcttccccgagtcctgtccagatttctgctgtggtacctgtgatgaccaatactgctgctctgacgtgctgaagaaatttgtgtggagcgaggaaaggtgtgctgtgcctgaggccagcgtgcctgccagtgtagagccggtggagcagctgggctcggcgctgaggtttcgccctggctacaacgaccccatgtcagggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 28 - DNA, 2337 bp, Homo sapiens
tctagctcagtcctggcccactgcgccagcgctgagcctgccagggctggggctggggatcaccttgggatgatggtgtcagtcccagggggcaggagatcgagtgtcctctgagctggcgactgggcctgtagaagggaaccggcatttgtggagtgtccactgagtgccaaggtctgcgctgggcactgtcctcgcaccgcctcacctagtcctcacgtagccctcgggcaagtgaggatccgccgggactgcggctgggagggatggctgtggctgtcccccagcccacacagtaggcgctcagtgtcagggtgcatattcccggggacgccctccaggcctgagagctgggggccgccgccgccccccatgcatccgcacgtggtgaggtgtgtatggcttcccgtggactcagcctcttccccgagtcctgtccagatttctgctgtggtacctgtgatgaccaatactgctgctctgacgtgctgaagaaatttgtgtggagcgaggaaaggtgtgctgtgcctgaggccagcgtgcctgccagtgtagagccggtggagcagctgggctcggcgctgaggtttcgccctggctacaacgaccccatgtcagggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 29 - DNA, 1904 bp, Homo sapiens
ctgtgtctttaagagggtggaacggggcttcgcgtctgtgcttcctgtggctgacgtcatctggaggagatttgctttctttttctccaaaaggggaggaaattgaaactgagtggcccacgatgggaagaggggaaagcccaggggtacaggaggcctctgggtgaaggcagaggctaacatggggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 30 - DNA, 2259 bp, Homo sapiens
cggacagaggttccgggaaccagccgggccggggcggggcggggcgagggagaggggcggccgcgcggatcactgaggctgtggcggcactgcgcccggcgctcgcgtccgtccgcccgtccgcccgcccagccatgactgcgccggtccccgcgccgcggatcctgttgccgttgctgttgctgctgctgctaacgccgcctccgggtgcacgtggtgaggtgtgtatggcttcccgtggactcagcctcttccccgagtcctgtccagatttctgctgtggtacctgtgatgaccaatactgctgctctgacgtgctgaagaaatttgtgtggagcgaggaaaggtgtgctgtgcctgaggccagcgtgcctgccagtgtagagccggtggagcagctgggctcggcgctgaggtttcgccctggctacaacgaccccatgtcagggggaggaaattgaaactgagtggcccacgatgggaagaggggaaagcccaggggtacaggaggcctctgggtgaaggcagaggctaacatggggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 31 - DNA, 2223 bp, Homo sapiens
tctagctcagtcctggcccactgcgccagcgctgagcctgccagggctggggctggggatcaccttgggatgatggtgtcagtcccagggggcaggagatcgagtgtcctctgagctggcgactgggcctgtagaagggaaccggcatttgtggagtgtccactgagtgccaagcacgtggtgaggtgtgtatggcttcccgtggactcagcctcttccccgagtcctgtccagatttctgctgtggtacctgtgatgaccaatactgctgctctgacgtgctgaagaaatttgtgtggagcgaggaaaggtgtgctgtgcctgaggccagcgtgcctgccagtgtagagccggtggagcagctgggctcggcgctgaggtttcgccctggctacaacgaccccatgtcagggggaggaaattgaaactgagtggcccacgatgggaagaggggaaagcccaggggtacaggaggcctctgggtgaaggcagaggctaacatggggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 32 - DNA, 2430 bp, Homo sapiens
tctagctcagtcctggcccactgcgccagcgctgagcctgccagggctggggctggggatcaccttgggatgatggtgtcagtcccagggggcaggagatcgagtgtcctctgagctggcgactgggcctgtagaagggaaccggcatttgtggagtgtccactgagtgccaaggtctgcgctgggcactgtcctcgcaccgcctcacctagtcctcacgtagccctcgggcaagtgaggatccgccgggactgcggctgggagggatggctgtggctgtcccccagcccacacagtaggcgctcagtgtcagggtgcatattcccggggacgccctccaggcctgagagctgggggccgccgccgccccccatgcatccgcacgtggtgaggtgtgtatggcttcccgtggactcagcctcttccccgagtcctgtccagatttctgctgtggtacctgtgatgaccaatactgctgctctgacgtgctgaagaaatttgtgtggagcgaggaaaggtgtgctgtgcctgaggccagcgtgcctgccagtgtagagccggtggagcagctgggctcggcgctgaggtttcgccctggctacaacgaccccatgtcagggggaggaaattgaaactgagtggcccacgatgggaagaggggaaagcccaggggtacaggaggcctctgggtgaaggcagaggctaacatggggttcggagcgaccttggccgttggcctgaccatctttgtgctgtctgtcgtcactatcatcatctgcttcacctgctcctgctgctgcctttacaagacgtgccgccgaccacgtccggttgtcaccaccaccacatccaccactgtggtgcatgccccttatcctcagcctccaagtgtgccgcccagctaccctggaccaagctaccagggctaccacaccatgccgcctcagccagggatgccagcagcaccctacccaatgcagtacccaccaccttacccagcccagcccatgggcccaccggcctaccacgagaccctggctggaggagcagccgcgccctaccccgccagccagcctccttacaacccggcctacatggatgccccgaaggcggccctctgagcattccctggcctctctggctgccacttggttatgttgtgtgtgtgcgtgagtggtgtgcaggcgcggttccttacgccccatgtgtgctgtgtgtgtccaggcacggttccttacgccccatgtgtgctgtgtgtgtcctgcctgtatatgtggcttcctctgatgctgacaaggtggggaacaatccttgccagagtgggctgggaccagactttgttctcttcctcacctgaaattatgcttcctaaaatctcaagccaaactcaaagaatggggtggtggggggcaccctgtgaggtggcccctgagaggtgggggcctctccagggcacatctggagttcttctccagcttaccctagggtgaccaagtagggcctgtcacaccagggtggcgcagctttctgtgtgatgcagatgtgtcctggtttcggcagcgtagccagctgctgcttgaggccatggctcgtccccggagttgggggtacccgttgcagagccagggacatgatgcaggcgaagcttgggatctggccaagttggactttgatcctttgggcagatgtcccattgctccctggagcctgtcatgcctgttggggatcaggcagcctcctgatgccagaacacctcaggcagagccctactcagctgtacctgtctgcctggactgtcccctgtccccgcatctcccctgggaccagctggagggccacatgcacacacagcctagctgcccccagggagctctgctgcccttgctggccctgcccttcccacaggtgagcagggctcctgtccaccagcacactcagttctcttccctgcagtgttttcattttattttagccaaacattttgcctgttttctgtttcaaacatgatagttgatatgagactgaaacccctgggttgtggagggaaattggctcagagatggacaacctggcaactgtgagtccctgcttcccgacaccagcctcatggaatatgcaacaactcctgtaccccagtccacggtgttctggcagcagggacacctgggccaatgggccatctggaccaaaggtggggtgtggggccctggatggcagctctggcccagacatgaatacctcgtgttcctcctccctctattactgtttcaccagagctgtcttagctcaaatctgttgtgtttctgagtctagggtctgtacacttgtttataataaatgcaatcgtttggagctgctgccccctttcttcctggcctcggctgctggaattggaatcaggctgtactctttccatccatttgggcttct

SEQ ID NO: 33 - DNA, 1567 bp, Mus musculus
tagattcgatttccttttctttgaaaggggaggagattgaaactgagtggcctctgatgaaaagaggggaagtcctgggctgcaggagccccttgagtgaaggcggaggctaacatggggttcggagcgaccgtcgccattggcgtgacaatctttgtggtgtttattgccactatcatcatctgcttcacctgctcctgctgctgtctgtataagatgtgctgcccccaacgccctgtcgtgaccaacaccacaactactaccgtggttcatgccccttaccctcagcctcaacctcaacctgtggcccccagctatcctggaccaacataccagggctaccatcccatgcccccccagccaggaatgccagcagcaccctacccaacgcagtacccaccaccctacctggcccagcccacagggccgccaccctaccatgagtccttggctggagccagccagcctccatacaacccgacctacatggattccctaaagacaattccctgaacctgcccccagcctctttggctgccatttatgtcgtgtgtgagtgagtgatacgcagagttctttactgctgtctgtggtgtgtgtgccttgtctagacatgtggcttcctctgctgttgaccaggtaggcgcaagtcttaccagtgtgggtcgggaccaacctgttttcttcctcacttgaaattgtactttctgaaatttcaagcaaattaaaaacaataaggtaggaggtatttcccacgtcacccaaggtgaccagccatggcctgtcatacttaggagagcaagcttttgcgggtacagagcagctttgggggtaaccagctagctgctgctaggcctttattcaccagggtttggctgcattggcagtgaggcaggtggctgggggtgacaccaggtgacaaggggactcagtgcagggggtcacaccacgcagaacaccatacactctccatcagctgtctgtctggatgtcactgtccttcccggggctgtatagagggccacatgtgttcactattcaggctccactgggggaattttcctacctttgctggcttggctcctgctcccaggccagggacctcggtctgtctactacacactctggtttctccctgcactgtctttttactgttagccaaacattttgcctgttttctgtctccagatgtgtgataattggtgtgaggttgaaatccctggttcctggaggacagacaacctgacctccgactgtcagtttcccttgacaccatcttcatagaaatacctgactcctgtaccacagtccagtttgtcccagtagcagggacaccaaggccaatgggttatctggaccaaaggtggggtggagggcctaggtggtatctccggcccagatgtgaatacctccatattccctgttggttcctgtttcactggctgttttagctttgtgttgattggtgtttctgagcattcagactccgcaccctcatttctaataaatgcaacattggacccgcttcccctttcttcagcgcctaggcagctggccttggctctac

SEQ ID NO: 34 - protein, 209 aa, Homo sapiens
MASRGLSLFPESCPDFCCGTCDDQYCCSDVLKKFVWSEERCAVPEASVPASVEPVEQLGSALRFRPGYNDPMSGFGATLAVGLTIFVLSVVTIIICFTCSCCCLYKTCRRPRPVVTTTTSTTVVHAPYPQPPSVPPSYPGPSYQGYHTMPPQPGMPAAPYPMQYPPPYPAQPMGPPAYHETLAGGAAAPYPASQPPYNPAYMDAPKAAL

SEQ ID NO: 35 - protein, 108 aa, Homo sapiens
MTAPVPAPRILLPLLLLLLLTPPPGARGEVCMASRGLSLFPESCPDFCCGTCDDQYCCSDVLKKFVWSEERCAVPEASVPASVEPVEQLGSALRFRPGYNDPMSGGGN

SEQ ID NO: 36 - protein, 77 aa, Homo sapiens [sequence omitted]

SEQ ID NO: 37 - protein, 137 aa, Homo sapiens
MGFGATLAVGLTIFVLSVVTIIICFTCSCCCLYKTCRRPRPVVTTTTSTTVVHAPYPQPPSVPPSYPGPSYQGYHTMPPQPGMPAAPYPMQYPPPYPAQPMGPPAYHETLAGGAAAPYPASQPPYNPAYMDAPKAAL

SEQ ID NO: 38 - protein, 132 aa, Mus musculus
MGFGATVAIGVTIFVVFIATIIICFTCSCCCLYKMCCPQRPVVTNTTTTTVVHAPYPQPQPQPVAPSYPGPTYQGYHPMPPQPGMPAAPYPTQYPPPYLAQPTGPPPYHESLAGASQPPYNPTYMDSLKTIP

SEQ ID NO: 39 - DNA, 3264 bp, Mus musculus
ccgggctggtattctcaacaataaaagaaactctggtggaatcaccatgcctgacattaagctgtactacaaagcaattgtgataaaaactgcatggtactggtacagtgacagacaggtagatcagtggaatagaattgaaggaccagaaatgaatccacacaactatggtcacttgatcaaaggggctaaaaccatccagtggaaaaaagaccgaattttcaacaaatggtgctgacacaactggcggttatcatgtagaagaatgcgaattgatccattcttatctccttgtacaaagctcaagtctaagtggatcaaagacctccacataaaaccaaagacactgaaattaatagaggagaaagtagggaaaagcctcgaagatatgggcacggggaaaaaattcctaaacagaacagcagtggcttgtgttgtaagatcaagcattgacaaatgggacctgataaaattgtaaagcttctgtaaggcaaaagacacttgtcaataagacaaaaaggccaccaacagattggaaaggatttttaccaatcctaaatctgataggggactaatatccaatatatacaaagagttcaagaagctgaactccagaaattcaaataaccccattaaaaatggggttcagagctaaacaaagaattctcaactgaggaataccgaatggctgagaagtacctgaaaaaaatgttcaacatccttaatcatcagggaaatgcaaatcaaaacaaccttgagattccacctcacaccagtcagaatggctaagatcaaaaactcaggtgacagcagatgctggagaggatgtggagaaagaggaacactcctccactgctggtgggattgcaagcttgtacaaccactttggaagtcagtctggaggttcctcagaaaattggacataatactaccagaagatccagcaatacctctcctgggcgtatacccagaagacgttccaactggtaataagaacacatcctccactatgttcatagcagcctttttataatagccagaagctgtaaagaacccagatgtccctcaacagaggaatggatacagaaaatgtggtacatttacacaatggagtactactcggctattaaaaacaatgaatttatgaaattcttggacaaatggatgtatctggaggatatcatccttagtgaggtaacccaatcacaaaagaagccattaggtatgcacccactgataagtggatattagcccagaaacatagaacacccaagctacaatttgcaaaacacaagaaaatcaagaagagggaagaccaatgaatgggtagatacttcattcctccttagactagggaacaaaatacccatgaaaggagttacagagacaaagtttggagctaagatgaaaggatggactattcagagactaccccacctggggatccatcccataatcagccaccaaacccagacactattgcatatgccagaaagattttgctgaagggaccctgttatagctgtctcgtatgaggctatgccagtgcctggcaaatacagaagtggatgctcacagtcatctataagatggaacacagggcccccaatggagaagctagagaaaacacccaaggagctgaaggggtctgcaatcctataggtggaacaacaatatgaactaaccagtacccccagagctcgtatctttagctgcatatgtagcagaagatggcctagtcggccatcactgggaagagagtccccttggtattgcaaactttatataccccagtataggggaatgccagggccaagaagcaggagttggtgggtaggggagcagggcggggggagggtatagggaacttttgggatagcatttgaaatgcatataaagaaaatatctaataaaaaattaaaaaaaaagattttgctgatatgaccctgatatagctgtctcttgtgaggctatgccagtgcctagccaatacagaagtggacgctcacagtctattggatagaacacaggacccccaataaaggagctagagaaagtacccaaaggagctaaaggggtctgcaaccctataggaggaacaatataaactaaccagtactcctgggcagtgcaagttcacattcctccgttccctggccttgttaggaactttgtcccacattgaggggaaggggcagcctgtgtgtaccctaggagctgttagttcttaactcaggattggatccctgagccagggtgagcagttacctggaggtggtcttggtcactggggagtgacattcctcagactgaggtcctggaaatctgctaaaagggacttgactttgttgagcaccattccctgcttcaagcagcgggatcctcctccggtgaatctgtgtgaggctccaaggagccagcaagaaaatcacagcttccctgagggcaaattggaggcgcgcaccagctgtacactgctggcgctcaagtgttaggataacgttgccacagagatccttttctagcacctaagtcagctgcaagggggggggggtctttccgcaaaagtatgcagtgagcagagggcaacttggatgcaccagccccttcctcctgcatctgggaaacctgtctcaaattttcgtggaccggtgctggagggactcccatggccaggctgggaggggcggccgcttcccttctcttccctcccacttcttccccctccctcccacctcctcccctctcgggcgggggttccggaaaccggccggggcggggcaaggaggctagggccgcgctggtcgcggaggttgcggcggcaccgtggtcttgggcttggtccgtctgttcgtccgtccgttggtctgtcccgccatggctgcgccggcgccctctctgtggaccctattgctgctgctgttgctgctgccgccgcctccgggtggtgagcctgggaggagggggcggtgtgtgctccctagggaccgggtcgggtcagacacttcccctagctgtctctggaatgagccagacaggaccagccttggagtgcttcgacttgactccctgaaaaccaggggggcgtctgcgaaccccaccactgacttcatcttgtcacctcttccctcctgagctgatactgactacctttgttgtgtccttcacggtgtgaaggccctacctattccaggggctggagcaagactcccgcctggggttagcttcaggctcaaagacagcctggcccttcccagcaaagac

(19) United States
(12) Patent Application Publication
(10) Pub. No.: US 2004/0146971 A1
(43) Pub. Date: Jul. 29, 2004

(54) Novel p53 inducible protein

(76) Inventors: David Philip Lane, Dundee (GB); Jean-Christophe Bourdon, Dundee (GB); Jochen Renzing, Dundee (GB)

Correspondence Address: MYERS BIGEL SIBLEY & SAJOVEC, PO BOX 37428, RALEIGH, NC, 27627, US

(21) Appl. No.: 10/469,626

(22) PCT Filed: Feb. 25, 2002
(86) PCT No.: PCT/GB02/00804

(30) Foreign Application Priority Data
Feb. 24, 2001 (GB) 0104588.9

(51) Int. Cl. C12Q 1/68; C07H 21/04; C07K 14/47

(52) U.S. Cl. 435/69.1; 435/320.1; 435/325; 530/350; 536/23.5

(57) ABSTRACT

The present invention relates to a protein which is induced by p53 and which promotes apoptosis. The present invention also relates to the gene encoding the protein as well as vectors and the like comprising the gene and also uses the gene/protein associated with promoting apoptosis.

The present invention relates to a p53 inducible protein which promotes apoptosis. The present invention also relates to the gene encoding the protein as well as vectors and the like comprising the gene and also uses of the gene/protein associated with promoting apoptosis.

Mutation of the p53 tumour suppressor protein is the most common genetic aberration known to occur in human cancers (Hollstein et al., 1991). The major consequences of such mutations are inactivation of the biological and biochemical functions of the p53 protein (Ko and Prives, 1996; Gottlieb and Oren, 1996; Levine, 1997; Oren, 1999). Wild-type p53 protein is involved in several biological functions such as replication, senescence, differentiation and DNA repair. The best described biological functions of p53 are the induction of cell cycle arrest and apoptosis in response to cellular stresses such as ionising radiation, UV radiation, serum starvation and hypoxia (Zhan et al., 1993; Kastan et al., 1991; Graeber et al., 1994). p53 may cause cell cycle arrest or apoptosis to prevent the accumulation of genetic damage, which can lead to neoplastic transformation. Hence p53 seems to function as a “guardian of the genome” (Lane, 1992).

The mechanisms by which p53 accomplishes its biological functions have not yet been completely defined. However, one of its most notable and well-documented biochemical properties is its ability to modulate gene expression (Ko and Prives, 1996; Gottlieb and Oren, 1996; Levine, 1997; Oren, 1999). p53 can act as a positive transcription factor which, in response to cellular stress, binds in a sequence-specific manner to DNA and induces the expression of genes containing such an element in their promoter or introns (El-Deiry et al., 1992; Funk et al., 1992; Bourdon et al., 1997). Only few genes (Waf, MDM2, GADD45, IGFBP3, Cyclin G, Bax, B99, PA26, KAI1, Fas, DR5/KILLER . . . ) (El-Deiry et al., 1993; Wu et al., 1993; Barak et al., 1994; Kastan et al., 1992; Buckbinder et al., 1995; Okamoto and Beach, 1994; Zauberman et al., 1995; Miyashita and Reed., 1995; Utrera et al., 1998; Velasco-Miguel et al., 1999; Mashimo et al., 1998; Munsch et al., 2000; Wu et al., 1997) are known to be directly transactivated, in vivo, by wild-type p53 after cellular stress. Identification of transcriptional targets of p53 is critical in discerning pathways by which p53 affects global cellular outcomes such as growth arrest and cell death. Identification of the cyclin-dependent kinase inhibitor Waf a p53-responsive gene helps to explain how p53 can induce cell cycle arrest (El-Deiry et al., 1993; Harper et al., 1993; Xiong et al., 1993). Nevertheless, several studies conducted on cells derived from Waf nullizigote (−/−) mice show that loss of Waf only partially abolishes the cell cycle arrests induced by p53 (Deng et al., 1995; Brugarolas et al., 1995), suggesting that other genes may be involved in this process. The p53 target genes B99 (Utrera et al., 1998) and 14.3.3σ (Hermeking et al., 1997; Chan et al., 1999) whose expression can induce a G2 cell cycle arrest may be such genes. In contrast, the biochemical basis of p53-mediated apoptosis is still unclear. Depending on the experimental models used, p53 transcriptional activity is required (Yonish-Rouach et al., 1996; Attardi et al., 1996) or dispensable (Caelles et al., 1994; Haupt et al., 1995) for p53-mediated apoptosis. Identification of the pro-apoptotic genes, Bax, Fas and DR5/Killer as p53 responsive genes, indicates that p53 transcriptional activity can play a role in p53 mediated-apoptosis. Studies conducted on cells derived from Bax−/− mice show that loss of Bax only partially abolishes the apoptotic function of p53 (Knudson et al., 1995; Yin et al., 1997) suggesting that other genes may be involved. Fas and KILLER/DR5 may be such genes but it rennins to be seen whether they play a key role in p53-dependent apoptosis. Tokino et al (1994), using a yeast-based assay, have estimated that the total number of p53 responsive elements in the whole human genome is between 200 to 300 suggesting that most p53 responsive genes have not yet been identified.

WO 00/78808 (Millennium Pharmaceuticals Inc.) describes several human and mouse secreted proteins. However, no definitive functions have been ascribed to them.

It is therefor amongst the objects of the present invention to seek to identify a novel pro-apoptotic p53-inducible gene.

Thus, an aspect of the present invention is to provide a nucleotide sequence encoding a gene responsive to p53.

Accordingly, the present invention provides an isolated nucleotide sequence encoding a p53-inducible protein as shown in FIGS. 2, 3, 13, 14, 15, 16, 17, 18 or 19, derivative or fragment thereof or species specific homologue thereof.

For the purposes of the description, the term “p53-inducible protein” refers to a protein whose mRNA expression and hence protein levels in a cell are increased above baseline levels when the p53 gene and, hence, protein is expressed.

Furthermore, “nucleotide sequence” will generally be referred to as DNA unless there is a different indication but is understood to be non-limiting and may include RNA, cDNA, etc.

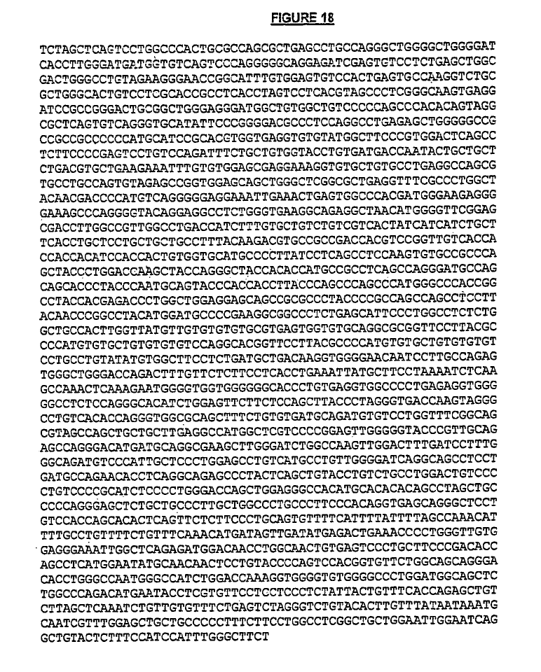

The present invention specifically provides an isolated nucleotide sequence encoding a p53-inducible protein from mouse (FIGS. 2 and 19) and human (FIGS. 3, 13, 14, 15, 16, 17 and 18).

The present inventors used the p53+/+ and p53−/− mouse model as a source of differentially expressed mRNA instead of cellular models in order to identify the p53-inducible gene/protein. Cellular models are generally established from tumour or immortalised cells that might have lost or reduced pro-apoptotic gene expression as an adaptation to in vitro culture. Hence, the present inventors compared the expression of genes in the spleen or thymus of normal and p53 nullizygote mice before and after γ-irradiation of whole animals and identified the p53-inducible protein by differential display. As will be described in more detail herein, the amino acid sequence and structure of the p53-inducible protein is conserved between human and mouse, and is subject to activation by p53 in both human and murine systems. Introduction of the cDNA suppresses growth of mouse or human tumour cells by promoting apoptosis independently of p53. Moreover, the protein is expressed in the endoplasmic reticulum and the nuclear envelope. N-terminal deletion mutants have lost pro-apoptotic activity and act in a dominant negative manner over wild-type protein. Inhibition of endogenous protein expression in NIH3T3-derived cells expressing antisense gene sequence increases resistance to apoptosis caused by DNA-damage or by impairment of the endoplasmic reticulum functions (ER stress). This novel gene therefore has all the characteristics expected of a gene that can contribute to p53-mediated apoptosis.

However, using the information provided by the present invention, a nucleotide coding sequence or a p53-inducible protein from any mammalian source may now be obtained using standard methods, for example, by employing consensus oligonucleotides and PCR. Furthermore, any promoter(s) associated with the p53-inducible gene may also be identified using information provided by the present invention.

The inventors have identified a number of splice variants resulting from the gene encoding the human form of the p53-inducible protein. The splice variants are illustrated in FIGS. 3, 13, 14, 15, 16, 17 and 18. The inventors have also identified a splice variant resulting from the gene encoding the mouse form of the p53-inducible protein, which is illustrated in FIG. 19. Therefore, the present invention is intended to cover these and other forms of splice variants.

The present invention also provides a nucleotide sequence which has 75% or above identity with the human nucleotide sequences disclosed herein, such as 76%, 80%, 83%, 86%, 90%, 93% or above. The term “Identity” as used herein can be readily calculated by known methods, including but not limited to those described in Computational Molecular Biology (Lesk, A. M., ed., Oxford University Press, New York, 1988), Biocomputing: Informatics and Genome Projects (Smith D. W., ed., Academic Press, New York, 1993), Computer Analysis of Sequence Data (Part I, Griffin, A. M. and Griffin, H. G., eds., Humana Press, New Jersey, 1994), Sequence Analysis in Molecular Biology (von Heinje G., Academic Press, 1987) and Sequence Analysis Primer (Gribskov M and Deveraux J., eds., M Stockton Press, New York, 1991; and Carillo, H., and Lipman, D., SIAM J. Applied Math. 48, 1073, 1988). The computer program method used to determine identity between two nucleotide sequences is BLAST which is publicly available from NCBI (www.ncbi.nlm.nih.gov) and other sources.

The present invention further provides a nucleotide sequence which has 98% or above identity with the mouse nucleotide sequences disclosed herein, for example, 99%.

Moreover, the invention also provides nucleotides complementary to those disclosed herein or sequences complementary to said nucleotide sequences for use in micro arrays, DNA arrays or DNA chips. These micro arrays may be useful for the determination from a biopsy of p53 activity and/or p53 responsiveness to cancer drug therapy.

In yet a farther aspect, the present invention provides use of the nucleotide sequences disclosed herein or sequences complementary to said nucleotide sequences for use in determining a loss of expression of the p53-inducible gene. Such a loss may be determined using techniques such as northern blot analysis, RT-PCR and other techniques known in the art.

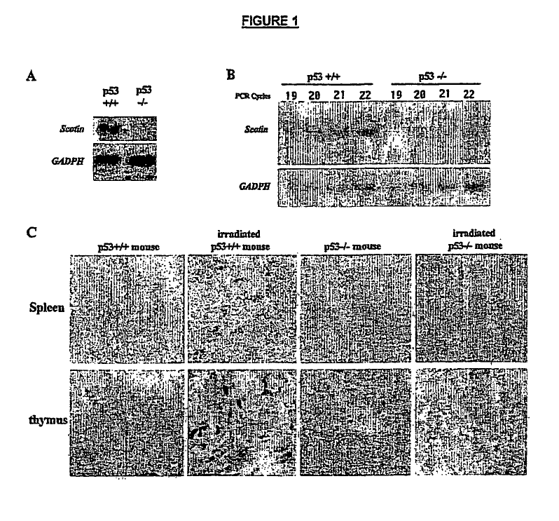

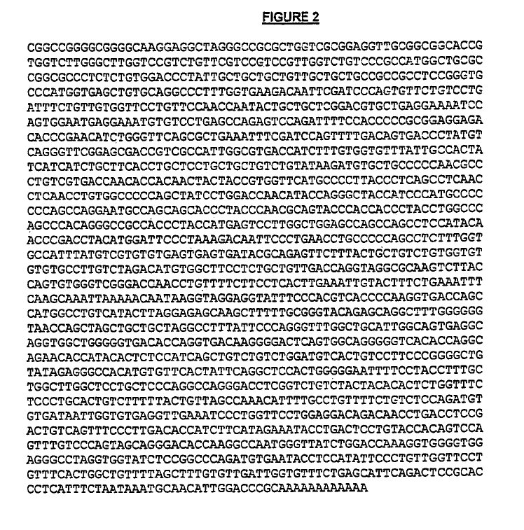

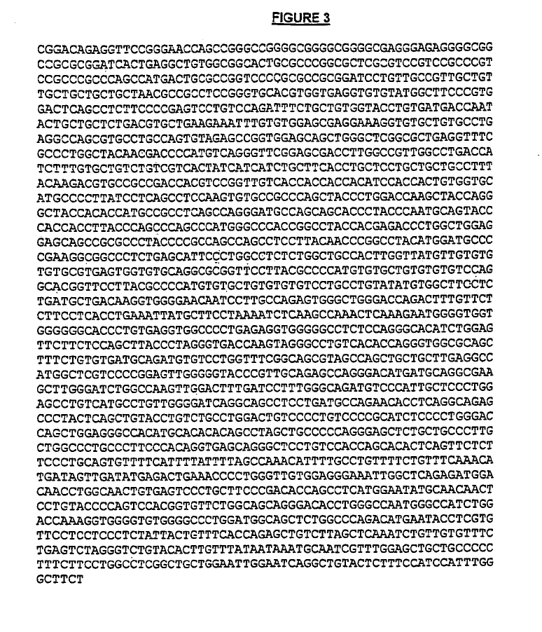

As is well known in the art, the degeneracy of the genetic code promotes substitution of bases in a codon resulting in a different codon which is still capable of coding for the same amino acid, a gene codon for amino acid glutamic acid is both GAT and GAA. Consequently, it is clear that for the expression of polypeptides with the amino acid sequences showing in FIGS. 4, 5, 20, 21, 22, 23, or 25, or fragments thereof, use can be made of derivative nucleic acid sequences with such an alternative codon composition different from the nucleic acid sequences showing in FIGS. 2, 3, 13, 14, 15, 16, 17, 18 or 19.

For recombinant production of the enzyme in a host organism, the nucleotide sequences encoding the p53-inducible protein may be inserted into an expression cassette is to form a DNA construct designed for a chosen host and introduced into the host where it is recombinantly produced. The choice of specific regulatory sequences such as promoter, signal sequence, 5′ and 3′ untranslated sequences, enhancer and terminator appropriate for the chosen host is within the level of skill of the routine worker in the art. The resultant molecule, containing the individual elements linked in a proper reading frame, may be introduced into the chosen cell using techniques well known to those in the art, such as calcium phosphate precipitation, electroporation, biolistic introduction, virus introduction, etc. Suitable expression cassettes and vectors and methods for recombinant production of proteins are well known for host organisms such as E. coli (see eg. Studier and Moffatt, J. Mol. Biol. 189: 113 (1986); Brosius, DNA 8: 759 (1989)), yeast (see eg. Schneider and Guarente, Meth. Enzymol 194: 373 (1991)) and insect cells (see eg. Luckow and Summers, Bio/Technol. 6: 47 (1988)) and mammalian cell (tissue culture or gene therapy) by transfection (Schenborn E T, Goiffon V. Methods Mol Bio. 2000; 130: 135-45, Schenborn E T, Oler J. Methods Mol Biol. 2000; 130: 155-64), electroporation (Heiser W C. Methods Mol Biol. 2000; 130: 117-34) or recombinant viruses (Walther W, Stein U; Drugs 2000 August; 60(2): 249-71).

Therefore, the invention further provides an expression cassette comprising a promoter operably linked to nucleotide sequence as disclosed herein encoding a p53-inducible protein or functionally active variant thereof.

In a yet further aspect, the present invention provides a nucleotide sequence comprising a transcriptional regulatory sequence, a sequence under the transcriptional control thereof which includes an RNA sequence characterised in that the RNA sequence is anti-sense to a mRNA which codes for p53-inducible protein.

The nucleotide sequence encoding the anti-sense molecule can be of any length provided that the anti-sense RNA molecule transcribable therefrom is sufficiently long so as to form a complex with a sense mRNA molecule encoding for p53-inducible protein. Thus, without the intention of being bound by theory, it is thought that the anti-sense RNA molecule complexes with the mRNA coding for the protein and prevents or substantially inhibits the synthesis of a functional p53-inducible protein. As a consequence of the interference by the anti-sense RNA, protein levels of p53-inducible protein are decreased or substantially eliminated.

The nucleotide sequence encoding the anti-sense RNA can be from about 20 nucleotides in length up to the length of the relevant mRNA produced by the cell. Preferably, the length of the nucleotide sequence encoding the anti-sense RNA will be from 50 to 1500 nucleotides in length. The preferred source of anti-sense RNA transcribed from DNA constructs of the present invention is nucleotide sequences showing substantial identity or similarity to the nucleotide sequence or fragments disclosed herein. The choice of promoter is within the skill of the person in the art, and may include a p53-inducible promoter.

The nucleotide sequence of the present invention may be employed using techniques in the art to obtain the promoter or regulatory nucleotides sequences to which the p53 protein binds. Thus, the present invention further provides use of the sequence disclosed herein for isolating and identifying a promoter and/or regulatory sequence(s) associated with the p53-inducible nucleotide sequences of the present invention.

The invention still further provides use of a sequence according to the present invention, whether “naked” or present in a DNA construct or biological vector, in the production of transgenic cells, particularly mammalian cells, having modified levels of p53-inducible protein. Recombinantly produced mammalian p53-inducible protein may be useful for a variety of purposes. For example, it may be used to investigate the role of the p53-inducible protein in vivo. Therefore, the present invention provides the recombinant production of the p53-inducible protein.

The present invention further provides a polypeptide substantially as shown in FIGS. 4, 5, 20, 21, 22, 23, or 25, derivatives or fragments thereof.

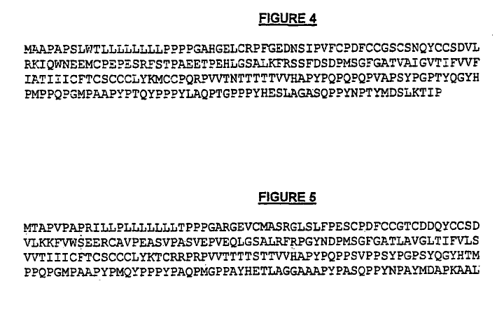

As discussed above, the inventors have identified a number of splice variants resulting from the gene encoding the human form of the p53-inducible protein. The proteins derived from these splice variants are illustrated in FIGS. 5, 20, 21, 22, and 23.

In addition, the protein derived from the alternative splice variant for the mouse form of the p53-inducible protein is illustrated in FIG. 25. Therefore, the present invention is intended to cover these and other forms of splice variants.

The present invention also provides a polypeptide sequence which has 67% or above identity with the human nucleotide sequences disclosed herein, such as 68%, 70%, 75%, 80%, 85%, 90%, 95%, 97% or 99% or above, or 74% similarity, such as 75%, 80%, 85%, 90%, 95%, 97% or 99% or above. The terms “identity” and “similarity” as used herein can be readily calculated by known methods, including but not limited to those described in Computational Molecular Biology (Lesk, A. M., ed., Oxford University Press, New York, 1988), Biocomputing: Informatics and Genome Projects (Smith D. W., ed., Academic Press, New York, 1993), Computer Analysis of Sequence Data (Part I, Griffin A. M. and Griffin, H. G., eds., Humana Press, New Jersey, 1994), Sequence Analysis in Molecular Biology (von Heinje G., Academic Press, 1987) and Sequence Analysis Primer (Gribskov M and Deveraux J., eds., M Stockton Press, New York, 1991; and Carillo, H., and Lipman, D., SIAM J. Applied Math. 48, 1073, 1988). The computer program method used to determine identity between two nucleotide sequences is BLASTP which is publicly available from NCBI (www.ncbi.nlm.nih.gov) and other sources.

The present invention further provides a nucleotide sequence which has 87% or above identity with the mouse nucleotide sequences disclosed herein, such as 88%, 90%, 95%, 97% or 99% or above, or 88% similarity, such as 89%, 90%, 95%, 97% or 99% or above.

Fragments are defined herein as any portion of the protein described herein that substantially retains the activity of the full-length protein. Derivatives are defined as any modified forms of the protein which also substantially retains the activity of the full-length protein. Such derivatives may take the form of amino acid substitutions which may be in the form of like for like eg. a polar amino acid residue for another polar residue or like for non-like eg. substitution of a polar amino acid residue for a non-polar residue as discussed in more detail below.

Replacement amino acid residues may be selected from the residues of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. The replacement amino acid residue may additionally be selected from unnatural amino acids. Within the above definitions of the peptide carrier moieties of the present invention, the specific amino acid residues of the peptide may be modified in such a manner that retains their ability to induce apoptosis, such modified peptides are referred to as “variants”, Thus, homologous substitution may occur i.e. like-for-like substitution such as basic for basic, acidic for acidic, polar for polar, etc. Non-homologous substitution may also occur ie. from one class of residue to another or alternatively involving the inclusion of unnatural amino acids such as ornithine (O), diaminobutyric acid (B), norleucine (N), pyriylalanine, thienylalanine, naphthylalanine and phenylglycine and the like. Within each peptide carrier moiety, more than one amine acid residue may be modified at a time, but preferably, when the replacing amino acid residue is alanine, less than 3.

As used herein, amino acids are classified according to the following classes;

basic; H,K,R

acidic; D,E

polar, A,F,G,I,L,M,P,V,W

non-polar, C,N,Q,S,T,Y,

(using the internationally accepted amino acid single letter codes) and homologous and non-homologous substitution is defined using these classes. Thus, homologous substitution is used to refer to a substitution from within the same class, whereas non-homologous substitution refers to a substitution from a different class or by an unnatural amino acid.

In general, the term “polypeptide” refers to a molecular chain of amino acids with a biological activity. It does not refer to a specific length of the products, and if required it can be modified in vivo and/or in vitro, for example by glycosylation, myristoylation, amidation, carboxylation or phosphorylation; thus inter alia peptides, oligopeptides and proteins are included. The polypeptides disclosed herein may be obtained, for example, by synthetic or recombinant techniques known in the art.

These terms also extend to cover for example, functional domains which may be observed in the protein and isolated polypeptides relating to these functional domains and which may be of particular use.

It will be understood that for the p53-inducible nucleotide and polypeptide sequences referred to herein, natural variations can exist between individuals. These variations may be demonstrated by amino acid differences in the overall sequence or by deletions, substitutions, insertions or inversions of amino acids in said sequence. All such variations are included in the scope of the present invention.

A further aspect of the present invention provides antibodies specific to the p53-inducible protein or fragment or derivatives thereof. Production and purification of antibodies specific to an antigen is a matter of ordinary skill, and the methods to be used are clear to those skilled in the art. The term antibodies can include, but is not limited to, polyclonal antibodies, monoclonal antibodies (mAbs), humanised or chimeric antibodies, single chain antibodies, Fab fragments, (Fab′)₂ fragments, fragments produced by a Fab expression library, anti-idiotypic (anti-Id) antibodies, and epitope binding fragments of any of the above. Such antibodies may be used in modulating the expression or activity of the full length p53-inducible protein or fragments or derivatives thereof or in detecting said polypeptide in vivo or in vitro.

It is postulated that antibodies to the p53-inducible protein or fragments or derivatives thereof may be present in the plasma of patients with cancer. Thus, the present invention further provides a method for the diagnosis of cancer in a patient, said method comprising the detection of antibodies to an abnormal form of the p53-inducible protein using the naturally occurring p53-inducible protein or fragments or derivatives.

It has been observed that mutation of the p53 tumour suppressor protein is the most common genetic aberration known to occur in human cancers. Major consequences of such mutants are inactivation of the biological and biochemical functions of p53. Therefore, it is envisaged that activation of genes which are induced by wild type p53 may promote apoptosis in cancer cells. It has been observed by the present inventors that the p53-inducible protein of the present invention appears to promote apoptosis independently of p53.

Therefore, in yet a further aspect, the present invention provides use of a nucleotide sequence encoding the p53-inducible protein, or fragments thereof, in the manufacture of a medicament for the treatment of diseases associated with abnormal proliferation of cells. Such diseases include cancer, eczema, and the like. The present invention also provides a method of treating diseases associated with abnormal cell proliferation comprising administering to a patient a therapeutic amount of p53-inducible protein in order to promote apoptosis in cells with abnormal proliferation.

Furthermore, the present invention provides a polypeptide which comprises the p53-inducible protein, or fragments thereof, in the manufacture of a medicament for the treatment of cancer. The treatment may include the topical application of the p53-inducible protein to surface tumours such as melanoma.

In yet a further aspect, the present invention provides use of the nucleotide and/or amino acids disclosed herein for the isolation and identification of agents, such as chemical compounds, which promote apoptosis by increasing expression of the protein and/or enhancing pro-apoptotic activity of the protein. Since the p-53 inducible protein is thought to be localised in the ER and nuclear membrane it is envisaged the agent may be additionally associated with a further compound(s) which assists in transporting the agent to the site of action. This may include compounds which enable the agent to cross the cell membrane to gain access to the ER and nuclear membrane.

The present invention also provides a method of treating diseases associated with abnormal cell proliferation comprising administering to a patient a therapeutic amount of an agent which promotes apoptosis in cells with abnormal proliferation by increasing the expression of the p53-inducible protein and/or enhancing pro-apoptotic activity of the protein. Such a treatment is understood to include the application of an adenovirus containing the p53-inducible nucleotide sequence coding for a functional p53-inducible protein. It is envisaged that the modified adenovirus may be injected into tumours where the p53-inducible protein is expressed and induces apoptosis in the tumour cells.

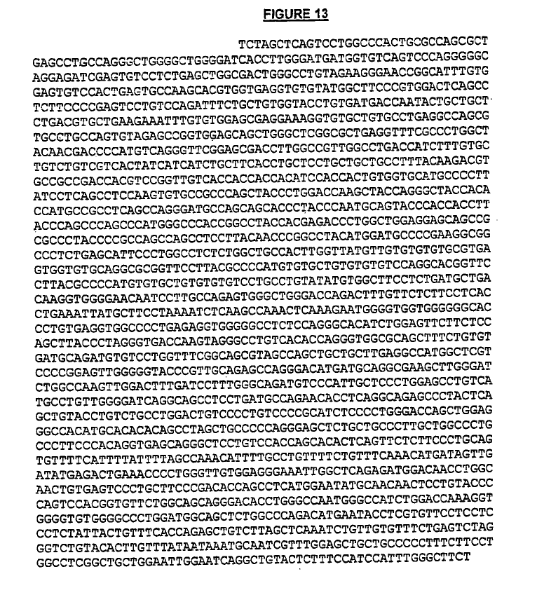

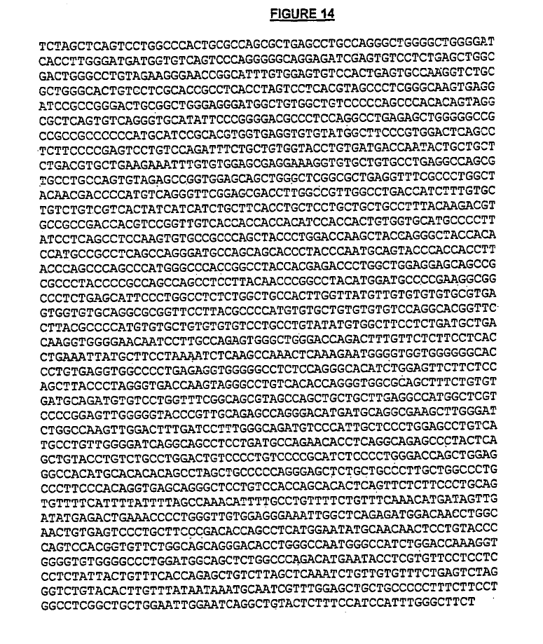

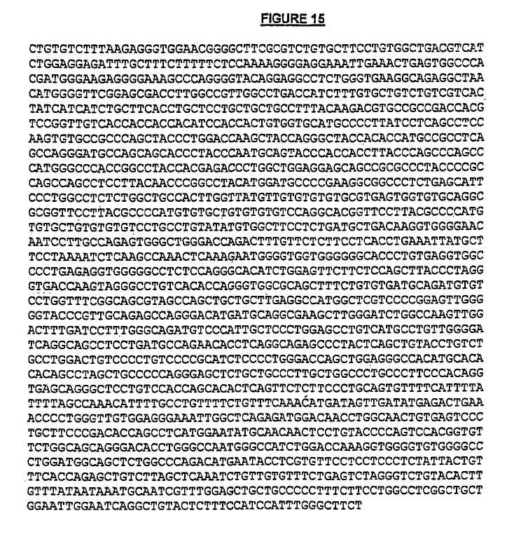

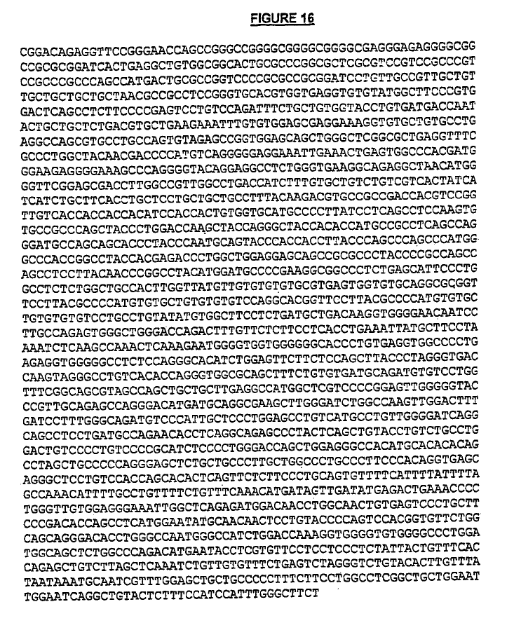

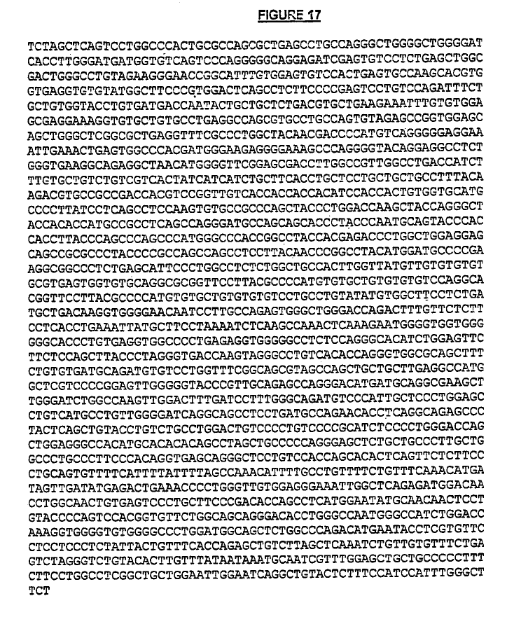

In a yet further aspect, the present invention provides a pharmaceutical formulation comprising a polynucleotide fragment comprising a nucleotide sequence of FIG. 2, FIG. 3, FIG. 3, FIG. 13, FIG. 14, FIG. 15, FIG. 16, FIG. 17, FIG. 18 or FIG. 19, or a fragment, derivative, or homologue thereof, and a pharmacologically acceptable carrier.

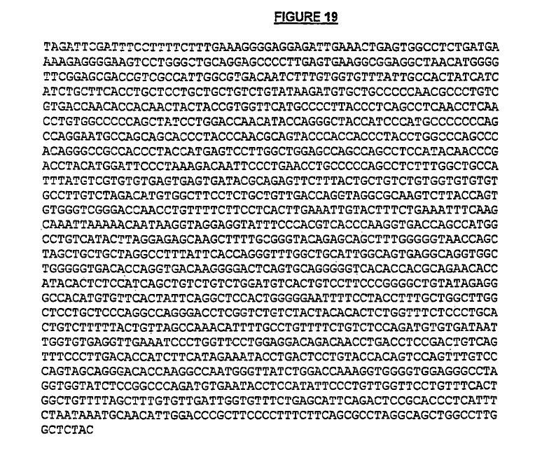

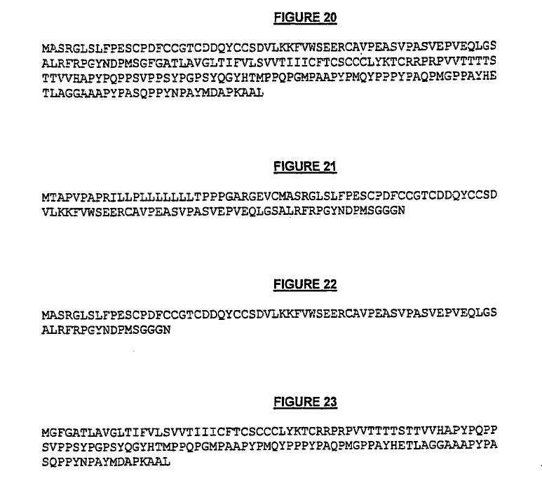

In a still further aspect, the present invention provides a pharmaceutical formulation comprising a polypeptide comprising an amino acid sequence of FIG. 4, FIG. 5, FIG. 20, FIG. 21, FIG. 22, FIG. 23 or FIG. 25, or a functionally active fragment, derivative, or homologue thereof, and a pharmacologically acceptable carrier.

These and other aspects of the present invention will become apparent from the following description when taken in combination with the accompanying drawings, in which:

FIG. 1 illustrates clone 105.9 (Scotin) mRNA being induced, in vivo, after γ-irradiation in spleen of normal mouse but not in p53−/− mouse.

p53 Deficient (−/−) mice as well as wild-type (WT) litter mates, were obtained through a cross between male and female p53+/− mice. One 6 weeks old mouse of each type was exposed to 5 Gy of whole body γ-irradiation. Total RNA was extracted 3 h later from the spleen of each mouse. a) Northern blot: 10 μg of total RNA was analysed by Northern blot with a mouse Scotin probe. After autoradiography, the blot was stripped and rehybridised with rat GADPH probe. b) semi-quantitative RT-PCR. 0.5 μg of total RNA were analysed by RT-PCT by incorporating ³³P-dATP and using Scotin specific primers or GAPDH specific primers as described in Experimental Procedure. PCR reactions were stopped after different cycles to assess the linear amplification. PCR products were electrophoresed on a 8% polyacrylamide gel before autoradiograph, c) In-situ hybridisation. Two p53+/+ male mice and two p53−/− male mice were exposed to 5 Gy of whole body γ-irradiation. Spleen and thymus were removed 3 h after irradiation and immediately frozen in liquid nitrogen. Cryosections of 5 μm were fixed in paraformaldehyde. Sections were incubated with a digoxigenin-labelled antisense Scotin RNA probe as described in Experimental Procedures. After washing, sections were incubated with anti-digoxigenin antibody conjugated to alkaline phosphatase. Scotin mRNA was then visualised by the addition of a precipitation substrate whose activity is revealed by adding a precipitating substrate (NBT/BCIP).

FIG. 2 illustrates the mouse cDNA sequence of Scotin.

FIG. 3 illustrates the human cDNA sequence of Scotin.

FIG. 4 illustrates the amino acid sequence derived from the cDNA sequence of FIG. 2.

FIG. 5 illustrates the amino acid sequence derived from the cDNA sequence of FIG. 3.

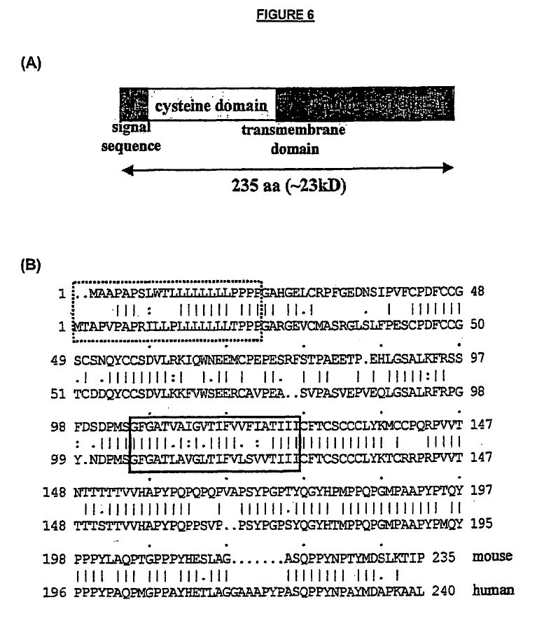

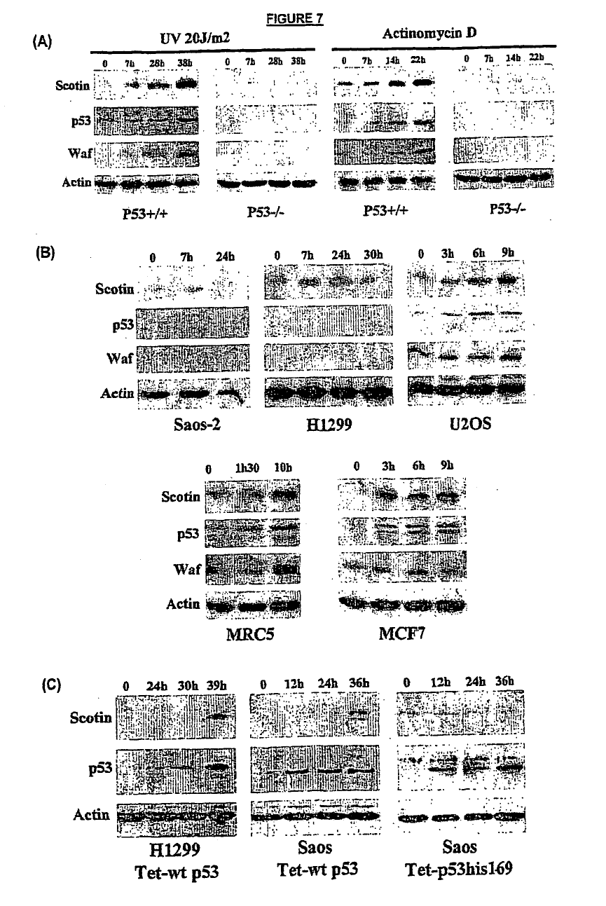

FIG. 6 illustrates a) schema of wild-type Scotin mouse protein primary structure, and b) human and mouse Scotin protein alignment with hydrophobic domain in solid box and a putative signal sequence in hashed box.

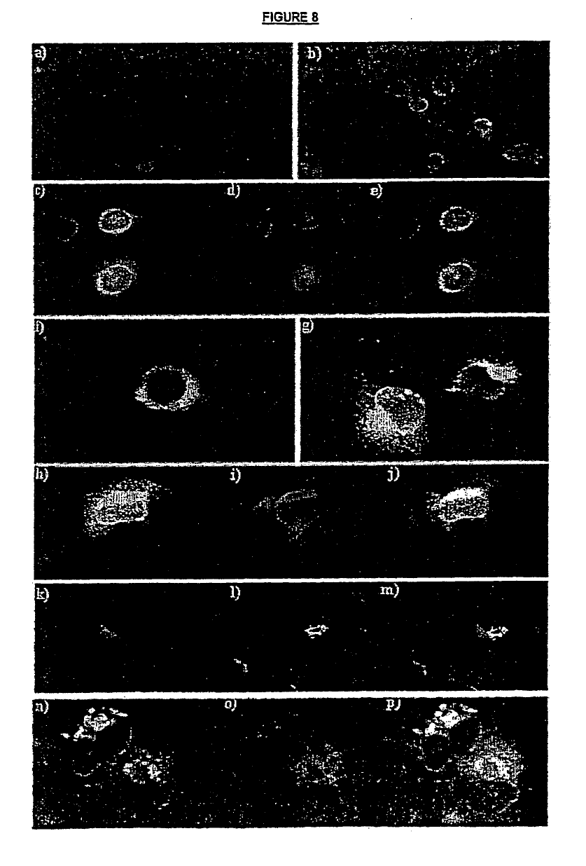

FIG. 7 is a western blot which illustrates that p53 is necessary and sufficient to induce Scotin protein expression. a) Only primary mouse embryonic fibroblasts (MEF) expressing WTp53 induce Scotin after UV irradiation or Actinomycin D treatment. MEF from p53−/− and p53+/+ littermate mice were exposed to UV-C light (20 J/m2) or Actinomycin D (60 ng/ml). Proteins were extracted at time indicated after treatment and analysed by Western blot by using affinity purified rabbit polyclonal anti-mouse-Scotin antibody. As a positive control, p53 and Waf induction were determined by using CM5 rabbit polyclonal anti-mouse p53 antibody and F5 mouse monoclonal anti-Waf antibody. To control loading and transfer efficiency, membranes were incubated with anti-actin mouse monoclonal antibody. b) Primary human fibroblasts and human tumour cell lines expressing functional p53 induce Scotin in response to Actinomycin D, a potent p53 activator. The primary human fibroblast MRC5, the tumour cell lines (MCF7, U2OS) expressing functional p53 and the tumour cell lines devoid of p53 expression (Saos-2, H1299) were treated with 60 ng/ml of Actinomycin D. Protein were extracted at time indicated and analysed by western-blot by using affinity purified rabbit polyclonal anti-human-Scotin antibody. c) p53 expression is sufficient to induce human Scotin expression. Proteins were extracted at times indicated after tetracycline induction from tetracycline-inducible p53 H1299 cells or Saos-2 cell lines described in Experimental Procedure. Scotin expression was analysed by Western blot by using affinity purified rabbit polyclonal anti-human Scotin antibody.

As a positive control, p53 and Waf induction were determined by using CM1 rabbit polyclonal anti-human p53 antibody and Ab1 mouse monoclonal anti-human-Waf antibody. To control loading and transfer efficiency, membranes were incubated with anti-actin mouse monoclonal antibody.

After incubation with the appropriate secondary anti-Ig conjugated to peroxidase, immunoblots were revealed by the ECL method.

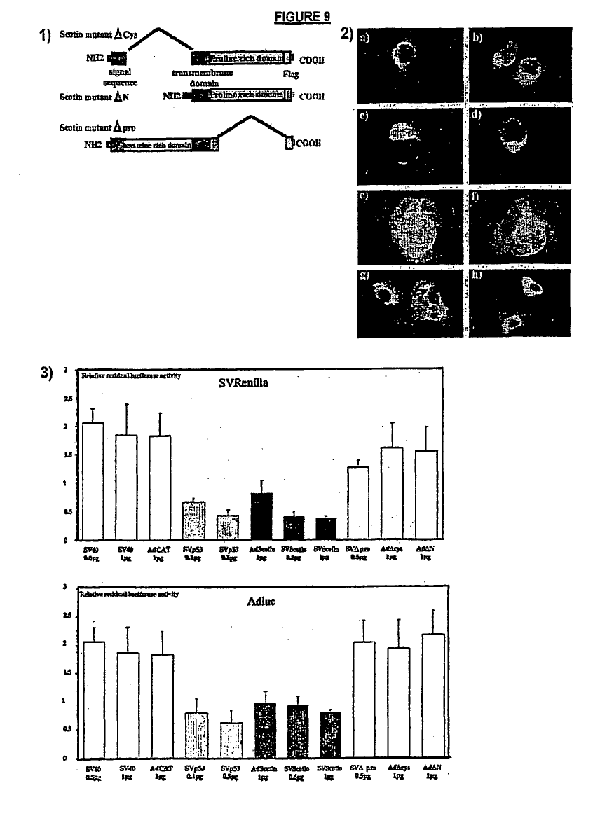

FIG. 8 illustrates that Scotin protein is expressed in the endoplasmic reticulum (ER) and the nuclear envelope. (a, b, c, d) Mouse and human endogenous Scotin proteins are expressed in the ER Mouse fibroblasts (3T3) and human tumour MCF7 cells (wt-p53) were exposed to 60 ng/ml of Actinomycin D and fixed after treatment. 3T3 cells were stained by indirect fluorescence (FITC) using anti-mouse Scotin antibody (a) 3T3 cells non-treated, (b) 3T3 cells treated. MCF7 were co-stained by indirect fluorescence using c) anti-human Scotin antibody (FITC) and d) the monoclonal anti-gp96 antibody (Texas-Red), e) Merge. gp96/GRP94 is a chaperon protein exclusively expressed in the ER. (f, g) Scotin is localised around the nucleus after ectopic expression. H1299 cells transfected with mouse Scotin expression vectors (f) 5 μg of AdScotin, (g) 10 μg of SVScotin, were stained by indirect fluorescence (FITC) using anti-mouse Scotin antibody. (h, i, j) Scotin is colocalised with gp96 in the ER after ectopic expression. H1299 cells transfected with 10 μg of SVScotin-flag expression vector, were co-stained by indirect fluorescence (h) using anti-Flag (M2) antibody (FITC) and (i) rabbit polyclonal anti-gp96 antibody (Texas-Red), (j) merge. (k, l, m) Scotin is not co-localised with TGN46 a marker of the Golgi apparatus. H1299 cells transfected with 5 μg of AdScotin-flag expression vector, were co-stained by indirect fluorescence using (k) anti-Flag (M2) antibody (Texas-Red) and (l) rabbit polyclonal anti-TGN46 antibody (FITC), (m) merge. (n, o, p) Scotin staining counterstained with mitochondria staining. H1299 cells transfected with 5 μg of SVScotin-flag expression vector, were co-stained by indirect fluorescence using (n) anti-Flag (M2) antibody (FITC) and (o) red mitotracker (Red), p) merge.

FIG. 9 illustrates that Scotin expression reduces constitutive luciferase expression after transfection.

1) Schema of the different Scotin mutants

2) Ectopic expression of Scotin mutants. Scotin mutants deleted of the carboxyl-terminus lose ER-localisation but the mutants deleted of the cysteine domain show ER localisation. H1299 cells were transfected with 0.5 μg of Adscotin-Flag (a) or SVscotin-Flag (b) or AdΔCys (c,d) or AdΔN (e, f) or SVΔpro (g, h). Cells were fixed and stained with anti-Flag (M2) mouse monoclonal antibody followed by anti-mouse antibody conjugated to FITC. 3) Cytotoxic assay based on the residual luciferase activity after transfection. Wild-type Scotin like p53 reduced constitutive luciferase expression but not Scotin deleted mutants. H1299 cells were co-transfected with SV40 Renilla luciferase (SVRenilla), AdMLP-luciferase (Adluc) and empty SV40 or SVp53 or Scotin expression vectors. The residual relative luciferase activity is calculated as described in the text. In case of inhibition of cell viability, the relative residual luciferase activity is expected to be inferior or equal to 1. (a) Histogram of the relative residual Renilla luciferase activity (SVRenilla). (b) Histogram of the relative residual Firefly luciferase activity (Adluc) (b). Histograms (a) and (b) represent the compilation of at least 3 independent experiments. Standard Deviation is reported as error bars.

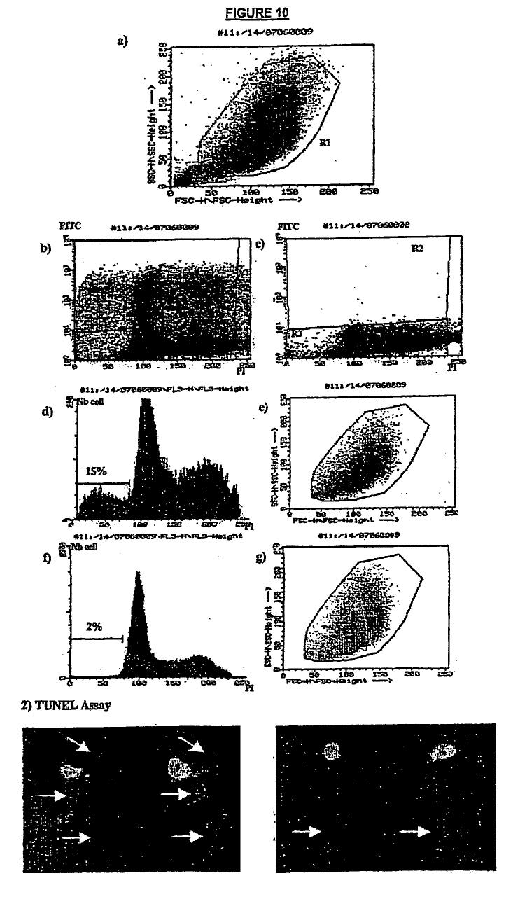

FIG. 10 describes the methods used to determine that Scotin induces apoptosis after transfection.

1) Three-parameter flow cytometry analysis. H1299 cells transiently transfected with AdCAT or SVScotin-Flag expression vectors were harvested 48 h after transfection, fixed and stained by indirect fluorescence (FITC) using anti-Flag antibody as described in Experimental Procedure. DNA was stained by propidium iodide (PI). To determine transfected cell population from the bulk of cells, we used a three-parameter flow cytometry analysis. (a) Cells were separated from cellular debris in function of size by gating the Forward Scatter versus Side Scatter dot plot (gate R1). (c) The non-transfected population was defined by gating the FITC versus PI dot plot (gate R3) obtained with AdCAT transfected cells indirectly stained with anti-Flag antibody. The transfected cells (gate R2) display a higher FITC intensity than the non-transfected cells. (b) The FITC versus PI dot plot obtained with SVScotin-Flag transfected cells indirectly stained with anti-Flag antibody, gate R2=transfected cells, gate R3=non-transfected cells.

(d) The DNA contents of the SVScotin-Flag transfected cells defined as the cells belonging to gate R1 and gate R2. (f) The DNA contents of the non-trasfected cells defined as the cells belonging to gate R1 and gate R3. The percentage of sub-G1 cells is indicated for each population. Events analysed in d) and f) are cells as assessed by the representation of the cellular size (e and g respectively)

2) TUNEL assay and immunostaining. Cells transfected by Scotin die by apoptosis. H1299 cells were transfected with 5 μg of Adscotin expression vector. Cells were fixed 48 h after transfection, subjected to TUNEL staining (left-hand grouping of cells of each image) and co-stained by indirect fluorescence using anti-mouse Scotin antibody (right-hand grouping of cells of each image) as described in Experimental Procedure. Cells stained by TUNEL were expressing Scotin. Arrows indicate non-transfected cells negative by TUNEL assay and not trasfected by Scotin.

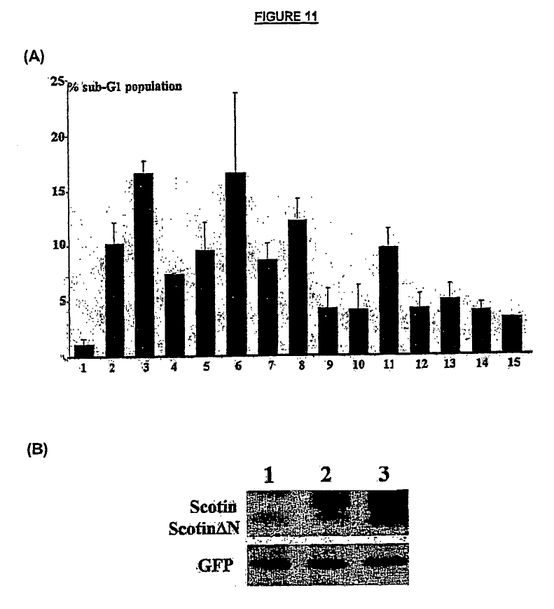

FIG. 11 illustrates that Scotin induces apoptosis after transfection

a) The DNA content of each transfected population was determined by three parameters flow cytometry analysis as described FIG. 10. The percentage of sub-G1 DNA content represents percentage of apoptotic cells. H1299 cells transfected or cotransfected with different expression vectors. 1: non transfected cells; 2: SVp53 0.5 μg/ml; 3: SVp53 2 μg/ml; 4: SVScotin 0.5 μg/ml; 5: SVScotin 2 μg/ml; 6: SVScotin 10 μg/ml; 7: AdScotin 1 μg/ml; 8: AdScotin 5 μg/ml; 9: AdΔCys 5 μg/ml; 10: AdΔN 5 μg/ml; 11: AdScotin 5 μg/ml and AdCAT 5 μg/ml; 12: AdScotin 5 μg/ml and AdΔCys 5 μg/ml; 13: AdScotin 5 μg/ml and AdΔN 5 μg/ml; 14: AdScotin 5 μg/ml treated with a cocktail of caspase inhibitors; 15: SVScotin 5 μg/ml treated with a cocktail of caspase inhibitors. Caspase inhibitor cocktail (10 μM) described in the Experimental Procedures was added 4 h before transfection. Histogram represents the average of at least three independent transfections. The Standard Deviation is reported as error bars.

b) Western-Blot: inhibition of Scotin mediated-apoptosis by Scotin mutant deleted of the N-terminus part is not due to inhibition of wild-type Scotin expression.

H1299 cells were cotransfected with 5 μg of AdΔN 5 μg and 5 μg of Adluc (lane1) or 5 μg of AdScotin-Flag and 5 μg of Adluc (lane2) or 5 μg of AdScotin and 5 μg of AdΔN (lane3). As a control for transfection efficiency, CMV-GFP (50 ng/ml) was included in each transfection mix. Scotin expression was revealed by western blot using anti-Flag monoclonal antibody. Transfection efficiency and protein loading were controlled by anti-GFP antibody.

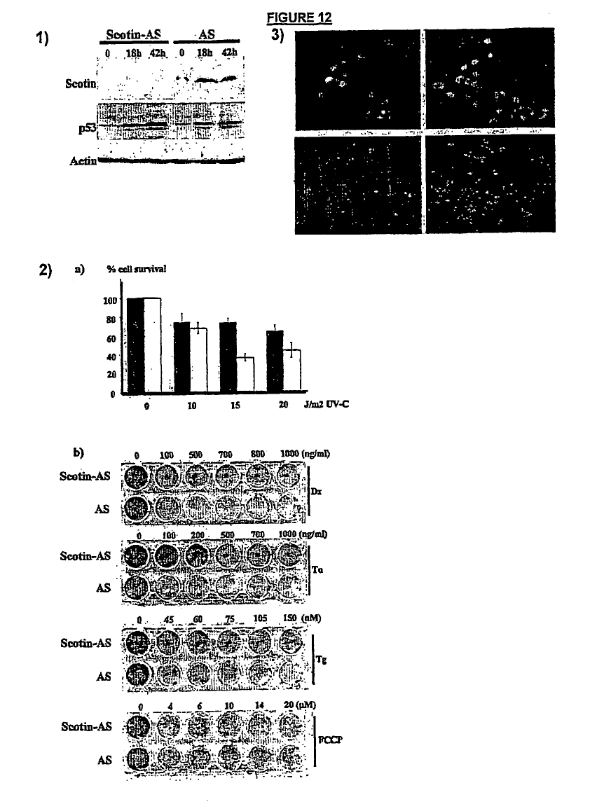

FIG. 12 illustrates that Scotin expression is required to induce apoptosis in response to DNA-damage and ER stress.

1) western-blot: Control antisense expressing cells (AS) and Scotin antisense expressing cells (Scotin-AS) were treated with Actinomycin D (60 ng/ml). Proteins were extracted at times indicated and analysed by western-blot. Scotin expression was revealed by anti-mouse scotin antibody. As a positive control, p53 induction was determined by using CM5 anti-mouse p53 antibody and protein loading was controlled by anti-actin antibody.

2) Scotin antisense expressing fibroblasts are resistant to apoptosis induced by DNA-damage and ER-stress. a) Cell survival assay: Scotin antisense (black) and control antisense (white) expressing fibroblasts were treated irradiated by UV-C at doses indicated. Cell survival was determinated as described in the Experimental Procedures by trypan blue 24 h after irradiation. Histogram represents the compilation of 4 independent experiments. Standard Deviation is reported as error bars. b) Clonoge assay; Scotin antisense and control antisense expressing fibroblasts were treated at concentrations indicated with the DNA-damaging agent Doxorubicin (Dx) or with the activators of ER stress Thapsigargin (Tg) or Tunicamycin (Tu) or with an activator of mitochondrial stress, FCCP. After treatment, cells were fixed in methanol and stained by Giemsa. Parental NIH3T3 cells behaved like the control antisense expressing cells after treatment by the same drugs.

3) NIH3T3 cells treated by tunicamycin die by apoptosis. NIH3T3 fibroblasts were treated for 24 h with 1 μg/ml of tunicamycin, fixed by paraformaldehyde and stained by TUNEL. Cells in apoptosis are stained by TUNEL (eft hand images). Similar results were obtained after treatment for 24 h with thapsigargin (150 nM).

FIG. 13 illustrates the cDNA sequence of a splice variant of Scotin (labelled Scotin2). This form of human Scotin cDNA starts from the alternative initiation site and is spliced in the first intron (the first exon of this form is not coding and the initiation site of translation starts in the second exon without changing the open reading frame).

FIG. 14 illustrates the cDNA sequence of a further splice variant of Scotin labelled Scotin2). This form of human Scotin cDNA starts from the alternative initiation site and is spliced in the first intron (the first exon of this form is not coding and the initiation site of translation starts in the second exon without changing the open reading frame).

FIG. 15 illustrates the cDNA sequence of a further splice variant of Scotin (labelled Scotin5). This form of human Scotia starts from the internal promoter encoding for scotin5.

FIG. 16 illustrates the cDNA sequence of a further splice variant of Scotin (labelled Scotin3).

FIG. 17 illustrates the cDNA sequence of a further splice variant of Scotin (labelled Scotin3). This form of human Scotin starts from the alternative initiation site of transcription.

FIG. 18 illustrates the cDNA sequence of a further splice variant of Scotin (labelled Scotin4). This form of human Scotin starts from the alternative initiation site of transcription.

FIG. 19 illustrates the cDNA sequence of a further splice variant of mouse Scotin starting from the internal promoter in intron 3.

FIG. 20 illustrates the amino acid sequence derived from the cDNA sequence of FIGS. 13 and 14.

FIG. 21 illustrates the amino acid sequence derived from the cDNA sequence of FIG. 16.

FIG. 22 illustrates the amino acid sequence derived from the cDNA sequence of FIGS. 17 and 18.

FIG. 23 illustrates the amino acid sequence derived from the cDNA sequence of FIG. 15.

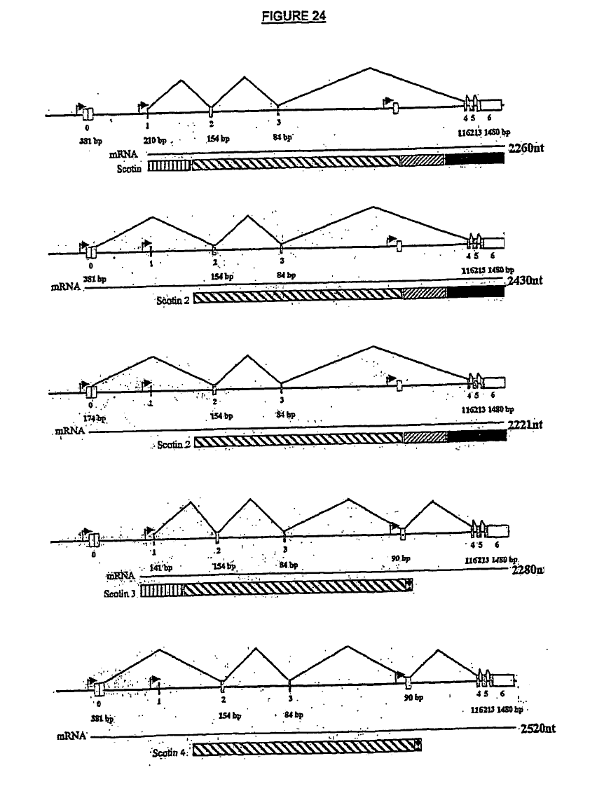

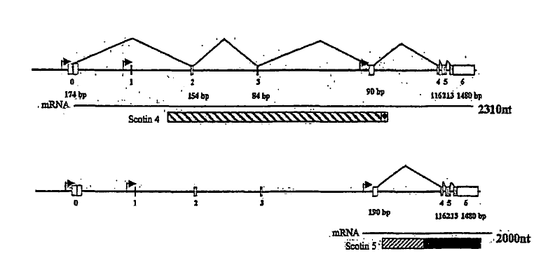

FIG. 24 illustrates the alternative splices and alternative initiation sites of transcription in the human Scotin gene. Coding exons are in grey, non-coding exons are in white. Arrows indicate the transcription sites. The lengths of the exons and mRNA are indicated. [symbol] denotes the signal sequences, [symbol] denotes the cysteine domain, [symbol] denotes the transmembrane domain, [symbol] denotes the proline/tyrosine domain and [symbol] denotes the 5 amino acids encoded by the alternative exon.

FIG. 25 illustrates the amino acid sequence derived from the cDNA sequence of FIG. 19.

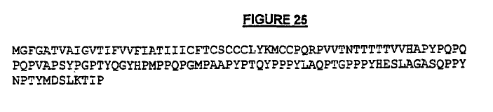

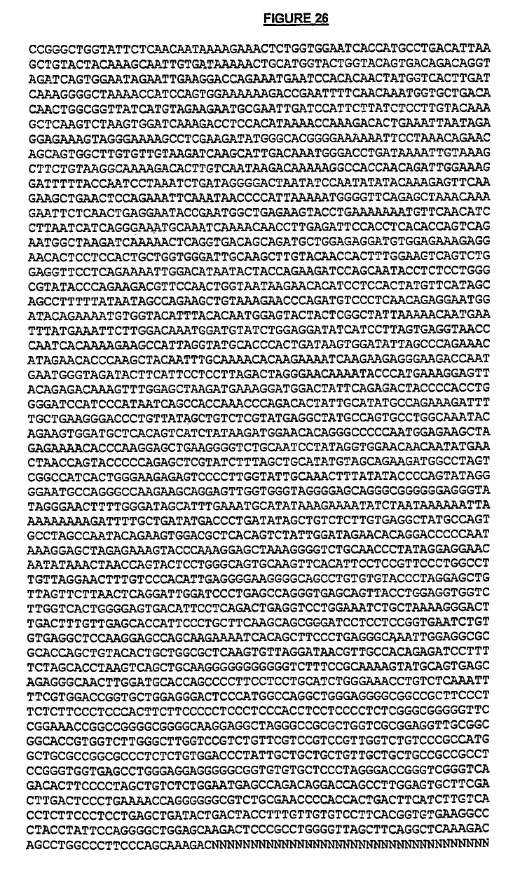

FIG. 26 illustrates the nucleotide sequence of the Scotin mouse promoter, which contains the p53 binding sites and is directly induced by p53.

### EXAMPLES

### Experimental Procedures

Cell Culture and Cellular Stress

All cell lines were purchased from ATCC except T22 (mouse fibroblasts) (Lu et al., 1996; Hupp et al., 1995) and p53−/− fibroblast (3T3) which were a gift from Dr. K. McLeod. U2OS (human osteosarcoma cell line expressing functional p53), T22, NIH3T3 cells (mouse fibroblast) and p53−/− mouse fibroblasts were maintained at 37° C., 5% C₂ in Dulbecco's modified Eagle's medium (DMEM supplemented with 10% heat-inactivated foetal calf serum (FCS). H1299, a human lung carcinoma cell-line devoid of p53, was routinely maintained at 37° C., 5% CO₂ in RPMI medium supplemented with 10% FCS.

H1299Tetwtp53 were derived from H1299 cells that were stably transfected with a tetracycline-inducible vector encoding for wild-type (wt) human p53 (Gossen et al., 1995). H1299Tetwtp53 cells were maintained at 37° C., 5% CO₂ in DMEM medium supplemented with 10% inactivated FCS, 0.4 mg/ml G418, 0.2 mg/ml hygromycin and 0.5 μg/ml anhydrotetracycline. To induce p53 expression, cells were washed twice with PBS and incubated with fresh medium supplemented containing no anhydrotetracycline. H1299Tetwtp53 cells were a generous gift from Dr. L. Debussche. SaosTetwtp53 and SaosTetmutp53 were derived from Saos-2 (human osteosarcoma cell lines devoid of p53) that were stably transfected with a tetracycline-inducible vector encoding for wt or mutant his169 mouse p53. Those cells were a gift from Dr. C. Midgley. Cells were routinely maintained at 37° C., 5% CO₂ in DMEM medium supplemented with 10% FCS and 0.5 mg/ml G418. To induce p53 expression, cells were washed twice with PBS and incubated with fresh medium supplemented with 10% FCS, 0.5 mg/ml G418 and 0.5 μg/ml anhydrotetracycline.

Scotin antisense cells were derived from NIH3T3 cells that were co-transfected in a stable manner with Scotin antisense expression vector (2.5 μg/ml) and Green fluorescent Protein (GFP) expression vector (5 ng/ml). Control antisense cells were derived from NIH3T3 cells that were transfected in a stable manner with pcDNA3 expression vector (2.5 μg/ml) and GFP expression vector (5 ng/ml). The pcDNA3 expression vector contains, between the CMV promoter and the poly (A) signal, a non-coding sequence of 100 bp not homologous to any known genes. We decided to use it without modification as a negative antisense control. Both cells lines were selected for 3 weeks in DMEM medium supplemented with 10% FCS and 0.5 mg/ml G418. GFP expression was used to assess cell transfection.

Actinomycin D (Sigma), solubilised in ethanol, was added to the culture medium at a final concentration of 60 ng/ml as described (Blattner et al., 1999). Prior to UVC irradiation, medium was removed and the cell layer was then irradiated with a UV-crosslinker (254 nm, 30 J/m²) and further cultured in the original conditioned medium. Thapsigargin and Tunicamycin were purchased from Sigma.

Differential Display, Dot-blot, RT-PCR and Northern-Blot

P53+/+ mice and p53−/− mice littermates (6 weeks old) were exposed for 1 min to 5 Gy of whole body γ-irradiation in a ¹³⁷Cs IBL 437C gamma irradiator. Spleen and thymus were resected 3 h following irradiation and immediately frozen in liquid nitrogen. Total RNA was extracted by using the kit ‘single step extraction reagents’ from Stratagen. RNA integrity was checked on an agarose gel for each sample before further analysis. The differential display was performed by using the “Delta™ RNA Fingerprinting” kit from Clontech in accordance with the manufacturer's protocol. After purification from dried polyacrylamide gel and reamplification by PCR, the differentially expressed DNA fragments from p53+/+ and p53−/− were cloned into a TA cloning vector from InVitrogen. As a differentially expressed DNA fragment can contain several different sequences, 10 colonies of each clone were analysed by dot-blot hybridisation to identify the true differentially expressed fragment(s).

Sequencing was performed by using DNA sequencing kit dRhodamine Terminator cycle sequencing (PE Applied biosystems) and T7 or SP6 primers. Sequences were analysed by ABI Prism 377 DNA sequencer.

Electrophoresis and Northern-Blot analysis were performed as previously described (May et al., 1989). The cloned differentially expressed fragments and a 1.3 kb PstI cDNA fragment corresponding to the rat GAPDH gene were used as radiolabelled probes for Northern-Blot analysis.

The semi-quantitative RT-PCR analysis was performed by using a poly-dT primer (18 mer) and the AMV reverse transcriptase followed by PCR using the mouse Scotin specific primer couple 5′-GCTGTATAGAGGGCCACATGTGTTCACT and 5′-AAAGACAGTGCAGGGAGAAACCAGAGTG or the mouse GAPDH specific primer couple 5′TGGACTGTGGTCATGAGCCC and 5′-CAGCAATGCATCCTGCACC. Scotin and GAPDH PCR products were electrophoresed on 8% PAGE/0.5% TBE before autoradiography.

In-situ Hybridisation

Two wt male mice and two p53−/− male mice were or were not γ-irradiated (5 Gy). Spleens and thymus were removed 3 h after irradiation and immediately frozen in liquid nitrogen. Cryosections of spleen and thymus (5 μm) were fixed in fresh 4% paraformaldehyde in PBS on ice, washed in sterile PBS and dehydrated in 25% Methanol/75% PBS then 50% Methanol/PBS and finally in 100% Methanol. The plasmid containing the differentially expressed figment was linearised and the antisense digoxigenin-labelled Scotin RNA was produced by T7 RNA polymerase and labelled with the ‘DIG RNA labelling’ kit from Roche Molecular Biochemicals. As a negative control, the sense digoxigenin-labelled Scotin RNA was produced by SP6 RNA polymerase. Sections were air-dried and overlaid with hybridisation solution containing antisense digoxigenin-labelled Scotin RNA probe. Sections were hybridised overnight at 60° C., washed at 55° C. in solution A (50% formamide, 2×SSC, 0.1% Tween 20), washed in TBS, and blocked 1 h at RT with 10% FCS in TBS. After incubation overnight at 4° C. with an anti-digoxigenin antibody conjugated to alkaline phosphatase (Roche Molecular Biochemicals) diluted 1/1500 in 1% FCS in TBS, the slices were washed with TBS and hybridised probe was revealed by hydrolysis of phosphatase substrate NBT/BCIP (Sigma).

Plasmids

The plasmid SVp53 is an expression vector of human wtp53 under the control of the SV40 early promoter (Nylander et al., 2000). The plasmid AdCAT encodes for the Chloramphenicol Acetyl Transferase driven by the minimal Adenovirus Major late Promoter (Ad) (Bourdon et al., 1997). The pAdluc plasmid was generated by cloning the Ad promoter sequence from AdCAT (XbaI/HindIII) upstream of the luciferase gene in pGL3-basic plasmid (Promega) (NheI/HindIII). The plasmid SVRenilla was purchased from Promega (pRL-SV40 vector). The empty plasmid SV40 was made by self-ligation of plasmid SVRenilla cut by NheI/XbaI.

Total mouse RNA extracted from thymus after ionising-radiation or human placenta mRNA (Clontech) were used as a source of mRNA in the 5′/3′ RACE lit (Roche Molecular Biochemical) using Taq polymerase (Expand™ high fidelity PCR system, Roche Molecular Biochemicals), to generate complete mouse and human Scotin cDNA. We designed primers from the sequence identified by differential display corresponding to the 3′end of mouse Scotin mRNA 5′-CCCGGGAAGGACAGTGACATC and 5′-TTCAAGTGAGGAAGAAAACAGG to extend to the transcriptional start site. The primer 5′-GGGCCTGCACAGCTCACCAT was used to extend to a position very close to the transcriptional start site. The mouse Scotin ORF was obtained by RT-PCR from total RNA extracted from mouse thymus after irradiation and the primer poly-dT (18 T) in the reverse transcription and then the primer couple 5′-CGGCCGGGGCGGGGCAAG and 5′-TCAGGGAATTGTCTTTAGGGAA. The amplified PCR product (942 bp) was cloned in TA cloning vector pTARGET Mammalian expression vector system from Promega to generate the plasmid (pTargetScotin). Five independent clones were sequenced.

Mouse Scotin expression vector (AdScotin) was constructed by ligating Scotin ORF from pTargetScotin (NheI/EcoRI), the intron contained in pTARGET plasmid (HindIII/EcoRI) into the Adluc plasmid (HindIII/XbaI). After sub-cloning, Scotin ORF sequence was checked by sequencing. PCR amplification using AdScotin plasmid as DNA source and the primer couple 5′-TATGTCAGGGTTCGGAGCGACCGTCGCCATTGG and 5′-CGCGCTCGAGCTACTTGTCATCGTCGTCCTTGTAATCGGGAATTGTCTTAGG was performed to add in frame the FLAG peptide to the carboxyl end of Scotin. The PCR product was cut by XhoI/BstXI and subcloned in AdScotin plasmid (XhoI/BstXI) to generate AdScotin-Flag plasmid. Scotin ORF fused to FLAG sequence was checked by sequencing.

SVScotin plasmid was generated by cloning the SV40 early promoter from SVRenilla plasmid (Promega) (KpnI/HindIII) and the intron-Scotin fragment from AdScotin (HindIII/BamHI) into AdScotin backbone plasmid (KpnI/BamHI). SVScotin-Flag was generated by cloning the SV40 early promoter from SVRenilla plasmid (KpnI/HindIII), the intron-mouse Scotin-Flag fragment from AdScotin-Flag (HindIII/BamHI) into the AdScotin backbone plasmid (KpnI/BamHI).

Mouse Scotin mutants, deleted of the N-terminus part, were made by PCR using the plasmid AdScotin-Flag as a source of DNA, the primer AVT7 5′-ACGACGTTGTAAAACGACGGCCAGAGAA with either the primer 5′-AGGCCGCGGGCGCAGCCATG to generate the mutant deleted of the entire N-terminus or the primer 5′-CAGACCGCGGGGATCGAATT to generate the mutant deleted of the cysteine rich domain. A SacII enzyme site present after the cysteine domain in mouse Scotin ORF was used to perform the mutants. Both PCR products were cut by EcoRI/SacII and cloned in the plasmid AdScotin-Flag cut by EcoRI/SacII to generate plasmids AdΔN and AdΔCys fused with Flag-peptide.

The mutant deleted of the proline/tyrosine domain was made by PCR using AdScotin plasmid as a DNA source and the primer couple 5′-TATGTCAGGGTTCGGAGCGACCGTCGCCATTGG and 5′-CGCGCTCGAGCTACTTGTCATCGTCGTCCTTGTAATCCAGACAGCAG. A XhoI site (underlined) and the Flag coding sequence were included in the last primer. The PCR product was cut by BstXI/XhoI and cloned in plasmid SVScotin-Flag cut by BstXI/XhoI to generate the plasmid SVΔpro.

We designed primers from the human EST Scotin sequence (GenBank AI040502) 5′-CTTCGCCGTTGGCCTGACCATCTT to extend to the 3′end of human Scotin mRNA and the primer 5′-CCACACTTGGAGGCTGAGGATAAGG to extend by RACE PCR using human placenta mRNA to a position close to the transcriptional start site. Both PCR products were cloned in TA cloning vector pGEM-T Easy (Promega) and 5 clones of each were sequenced.

The mouse Scotin cDNA fragment cloned in the antisense orientation into the pcDNA3 expression vector was obtained by PCR using the AdScotin plasmid as DNA template and by the primer couple 5′-GCCCTCGAGCCTCCGGGTGCCCATG and 5′-GCGGAATTCGCGGGGGTGGAAAATCTG. All constructs were checked by sequencing.

Cytotoxic assay based on luciferase activity: 3×10⁵ H1299 cells were seeded per well of four 24-well plates. Cells were co-transfected in duplicate per plate by calcium phosphate precipitate with a transfection mix (100 μl) containing Adluc (0.1 μg) and SVrenilla (0.2 μg) and plasmids indicated in the legend of FIG. 10. The total DNA in each transfection mix was balanced to 20 μg/ml by using pBluescript plasmid. After 6 h incubation at 37° C. in the presence of the DNA precipitate, cells were washed before further incubation at 37° C. The 24-well plates were harvested 18 h, 28 h, 42 h and 52 h after addition of the DNA precipitate. Cells were washed and lysed directly by adding 50 μl/well of passive lysis buffer 1X provided in the ‘Dual Luciferase Reporter Assay’ kit (Promega). After incubation at RT, 20 μl of each cell extract are transferred in a 96-well microplates (Falcon 3296) to be analysed in a Microlumat LB 96V luminometer (Berthold EG&G Instrument). The dual luciferase reporter assay (Promega) was performed according to the manufacturer's protocol.

Facscan analysis: 8×10⁵H1299 cells were seeded in a 10 cm Petri dish and transfected with 1 ml of calcium phosphate precipitate containing the plasmids as indicated in Table 1 (see page 45.). The total DNA in each transfection mix was balanced to 20 μg/ml by using pBluescript plasmid. After 16 h incubation at 37° C. in the presence of the DNA precipitate, cells were washed before incubation at 37° C. for a further 32 h. Cells were trypsinised 48 h after transfection, fixed in 70% ethanol and immunostained as described (Yonish-Rouach et al., 1994). Scotin transfected cells were stained by monoclonal anti-Flag antibody (3 μg/ml) followed by anti-mouse antibody conjugated to FITC (dilution 1/60). p53 transfected cells were stained by the monoclonal anti-p53 DO-1 antibody (1 μg/ml) followed by anti-mouse antibody conjugated to FITC (dilution 1/60). AdCAT transfected cells were stained by anti-Flag antibody (3 μg/ml) followed by anti-mouse antibody conjugated to FITC (dilution 1/60) to define the background of both antibodies in Facscan analysis. DNA was stained just before analysis by propidium iodide (12 μg/ml supplemented with RNAse A). 10⁵ Cells were analysed by flow cytometry (Facscan, Becton Dickinson) using a three-parameter analysis. Experiments presenting less than 2% of transfection efficiency were discarded.

In the experiments using a cocktail of caspase inhibitor, each caspase inhibitor (Z-DEVD-FMK, Ac-YVAD-CHO and Z-VAD-FMK, Calbiochem) was added to a final concentration of 10 μM final 4 h before transfection to the culture medium and maintained during the cell incubation.

Western Blot

Cells were washed and scraped in PBS buffer and centrifuged at 2000 rpm for 2 min. The cell pellet was lysed in 50 μl of RIPA buffer (PBS, 1% NP-40, 0.5% sodium deoxycholate, 0.1% SDS and 1 mM Protease Inhibitor cocktail) and incubated on ice for 30 min Protein extracts were centrifuged at 30,000 g for 20 min at 4° C. Protein concentration of the supernatant was determined by the Bradford assay. For each assay, a volume of supernatant corresponding to 50 μg of total protein (unless otherwise mentioned) was denatured for 5 min at 95° C. in Laemmli buffer and separated by electrophoresis on 15% SDS-PAGE. After migration, proteins were electrotransferred onto nitrocellulose membrane (PROTRAN®, Schleicher & Schuell). Transfer efficiency was estimated by Ponceau Red staining. Membrane was incubated for 30 min at RT in 10 ml PBS containing 0.1% Tween and 5% skimmed powder milk (PBSTM). Primary antibody was diluted in PBSTM and incubated with the-membrane for 1 h at RT. Finally, the corresponding HorseRadish Peroxidase-conjugated anti-mouse (Dako p0161) or anti-rabbit (DAKO p0217) immunoglobulin diluted 1:1000 in PBSTM was incubated with the membrane for 1 h at RT. The Western blots were revealed by ECL method (Amersham).

Immunofluorescence Staining

Cells (3×10⁵) were seeded on 2-well glass chamber slides (Lab Tek chamber slide, Cat.# 177380). Cells were transfected as previously described, fixed by 4% paraformaldehyde in PBS (unless otherwise mentioned) and permeabilised for 2 min at 4° C. in 0.1% Triton X-100, 0.1% citrate sodium Cells were then incubated for 1 h at RT with the primary antibody diluted in 10% FCS-DMEM. Cells were washed with PBS and incubated with Fluorescein (FITC)-conjugated donkey anti-rabbit IgG (Jackson Immunochemicals) or Fluorescein (FITC)-conjugated donkey anti-mouse IgG (Jackson Immunochemicals) diluted 1:200 in 10% FCS-DMEM depending on the primary antibody used. For double immunofluorescence staining. Fluorescein (FITC)-conjugated donkey anti-mouse IgG (Jackson Immunochemicals) diluted 1:200 in 10% FCS-DMEM and Texas-Red-conjugated goat anti-rabbit IgG (Jackson Immunochemicals) diluted 1:500 in 10% FCS-DMEM were used.

For TUNEL assay, cells (3×10⁵) seeded on 2-well glass chamber slide, were transfected as described. Cells were fixed for 30 min at RT in 4% paraformaldehyde in PBS, washed in PBS and permeabilised 2 min at 4° C. in 0.1% Triton X-100, 0.1% sodium citrate. The TUNEL staining was performed accordingly to the manufacturer's protocol (In Situ Cell Death Detection kit, Roche Molecular Biochemicals). The apoptotic cells presenting fragmented DNA were then labelled in green after incorporation of fluorescein. Immunostaining for Scotin expression was performed as previously described and revealed by using Texas-Red-conjugated goat anti-rabbit IgG (Jackson Immunochemicals) diluted 1:500 in 10% FCS-DMEM.

After immunostaining, cells were washed in PBS and stained with DAPI; 0.5 μg/ml, (Sigma) (unless otherwise mentioned) for 5 sec and washed with PBS. The cells were visualised by confocal microscopy.

Production and Affinity Purification of the Mouse and Human Anti-Scotin Antibodies

The peptide PYHESLAGASQPPYNPTYK, corresponding to the end of mouse Scotin protein, or the peptide YHETLAGGAAAPYPASQPPK, corresponding to the end of human Scotin protein, were conjugated to the carrier protein KLH and inoculated to a rabbit as described in the manual ‘Antibodies a laboratory manual’ by Ed Harlow and David Lane.

The anti-Scotin antibodies were purified by affinity purification using a peptide column. The antibody concentration was determined by the Bradford method.

Antibody: The anti-p53 rabbit sera (CM1 and CM5) were described in Midgley et al., 1992 and Midgley et al., 1995, the anti-p53 DO-1 mouse monoclonal antibody was described in Stephen et al., 1995. The rabbit polyclonal anti-TGN46 antibody was described in Prescott et al., 1997. The rabbit polyclonal anti-calnexin antibody was purchased from StressGen Biotechnologies Corp. The rabbit polyclonal anti-gp96/GRP94 antibody is a generous gift from Dr. T. Wileman. PC-10 antibody is a monoclonal anti-PCNA (Proliferating-Cell Nuclear Antigen) (Waseem and Lane, 1990). The mouse monoclonal anti-Flag antibody was purchased from Sigma (anti-Flag® M2 monoclonal). The mouse monoclonal (F-5) anti-Waf antibody was purchased from Santa-Cruz. The IgM mouse monoclonal Anti-Actin antibody (Actin Ab-1) was purchased from Calbiochem. The mouse monoclonal anti-α-tubulin was purchased from Amersham.

### Results

Isolation of a Novel p53-regulated Gene by Differential Display

Previous studies have shown that cells from thymus or spleen undergo massive p53-dependent apoptosis after γ-irradiation in normal mice but not in p53 nullizygote mice (Lowe et al., 1993; Clarke et al., 1993; Midgley et al., 1995). This model can therefore be used to identify pro-apoptotic genes induced, in vivo, by p53 after γ-irradiation of the entire animal. With this aim, we have compared by differential display the expression of genes in spleen or thymus of normal and p53 nullizygote mice (Donehower et al., 1992) after γ-irradiation of whole animals.

Two female mice, one p53−/− and the other p53+/+ from the same litter (6 weeks old), were γ-irradiated for 1 min at a dose of 5 Gy/min in a ¹³⁷Cs gamma irradiator. The spleen and thymus were removed 3 h after irradiation and frozen immediately in liquid nitrogen. After total RNA extraction from spleens, the two RNA populations from the p53+/+ and the p53−/− irradiated mice were subjected to screening by a differential display method (Liang and Pardee, 1992; Zhao et al., 1996). To identify genes specifically induced by p53 in response to irradiation, we compared only expression of RNA from p53+/+ and p53−/− irradiated mice. Hence, the genes induced in response to irradiation but independently of p53 did not appear differentially expressed. The screening resulted in the isolation of 112 short PCR-amplified DNA fragments that were differentially expressed. Forty-six fragments among the most differentially expressed were cloned. As some of the isolated fragments consisted of several different sequences of the same size, 10 subclones of each fragments band were tested in a duplicate dot-blot hybridisation to identify those corresponding to true differentially expressed transcripts.

After isolation, each true differentially expressed transcript was sequenced. Sequences corresponding to the Ig Heavy chain genes, whose expression is already known to be p53 dependent (Shaulsky et al., 1991), were found in several clones. However, the sequences were new for most of the other differentially expressed transcript clones, suggesting that they represent novel genes.

We analysed mRNA levels of 10 of the most differentially expressed mRNAs by Northern blot and semi-quantitative RT-PCR to confirm differential expression, comparing levels after irradiation in spleens from normal or p53−/− mouse. Clone 105.9 displayed stronger and more consistent induction after ionising radiation in the wild-type mouse than in p53−/− mouse (FIGS. 1a and b) suggesting that the differential expression was p53-dependent and not only irradiation-dependent. Clone 105.9 was therefore chosen for further study and was named Scotin.

In order to confirm the in vivo differential expression of Scotin mRNA, we performed an in situ hybridisation analysis (FIG. 1c). Both wt and p53−/− male mice were γ-irradiated (5 Gy). Spleens and thymus were resected 3 hours after irradiation along with the same organs from non-irradiated mice of the same genotype as controls. Cryosections of spleen and thymus were treated and hybridised with an antisense digoxigenin-labelled Scotin RNA probe. After incubation with an anti-digoxigenin antibody conjugated to alkaline-phosphatase, the hybridised probe was revealed by hydrolysis of phosphatase substrate NBT/BCIP. FIG. 1c shows that Scotin mRNA was strongly induced only after radiation in the spleen and in the thymus of the wt mice. However, all cells did not induce Scotin mRNA after irradiation probably because p53 is not homogeneously expressed in vivo after cellular stress (Hall et al., 1993; Lu and Lane, 1993; Komarova et al., 1997). No induction of Scotin mRNA could be detected in the spleen or thymus of p53−/− mice after γ-irradiation. Hybridisation with the sense-digoxgenin-labelled Scotin RNA probe performed in the same conditions gave no signal, confirming that the in situ hybridisation was specific for Scotin mRNA (data not shown). Altogether, these results indicate that Scotin gene expression is induced, in vivo, in a p53-dependent manner in response ionising radiation.

Scotin Gene is Conserved Between Mouse and Human

We designed primers from the short sequence identified by differential display corresponding to the 3′end of mouse Scotin mRNA to perform a 5′/3′ RACE PCR with mRNA extracted from thymus of irradiated p53+/+ mice in order to extend to a position very close to the transcriptional start site. We obtained a sequence of 1850 bp consistent with the apparent size of Scotin mRNA observed in Northern-Blots. The sequence contains a short 5′ untranslated region (5′UTR), only one open reading frame (ORF) and a relatively short 3′ UTR (FIG. 2). The presence of an in-frame stop codon within the 5′UTR supports the correct assignment of the first methionine of the ORF. This ORF predicts a protein of 235 amino acid residues, containing in the N-terminus a putative signal sequence of 22 residues immediately followed by a domain rich in cysteine. In the central part of the protein are 18 hydrophobic residues corresponding to a putative transmembrane domain and at the carboxy terminal end there is a domain rich in proline and tyrosine. (FIG. 6a). No further protein domain homologies have been identified to any known gene product.

We searched in Genbank for mouse and human EST sequences (dbEST database) homologous to mouse Scotin cDNA. Two sets of mouse EST sequences homologous to mouse Scotin could be defined, one encompassing the EST sequences identical to mouse Scotin cDNA and another set of EST sequences containing a different 5′end to mouse Scotin cDNA. The latter set may therefore represent a Scotin-related gene. We also identified two sets of EST sequences in human homologous to the two sets previously identified in mouse suggesting that the Scotin gene belongs to a conserved family of genes. None of the EST sequences identified contained a complete ORF.

To obtain human Scotin cDNA, we designed primers from the longest human EST sequence homologous to mouse Scotin, AI040502, and performed a 5′/3′ RACE PCR on mRNA from human placenta. We obtained a complete cDNA with the apparent size of 2 kb (see FIG. 3). It contains one ORF and a relatively long 3′ UTR. The ORF predicts a protein of 239 amino acid residues sharing 72% homology (70% identity) with the mouse Scotin protein (FIGS. 5 and 6b). Alignment of both Scotin proteins (FIG. 6b) shows that the signal sequence, the cysteines in the N-terminus, the hydrophobic and the proline/tyrosine domains are well conserved.

Scotin Protein Expression is Induced in a p53-dependent Manner in Response to Cellular Stress

Two affinity purified rabbit polyclonal antibodies, JC105 and H105 were raised against a peptide corresponding to the carboxyl-end of mouse or human Scotin protein respectively. Their respective specificity was assessed by Western blot analysis using mouse or human Scotin protein produced by an in vitro coupled transcription/translation assay. Mouse and human anti-Scotin antibody detected only one protein with an apparent size of 25 kDa consistent with the expected size for Scotin proteins (data not shown).

In order to further characterise Scotin protein, it was essential to identify cell lines that could induce Scotin upon DNA damage. We exposed to UV-C light or Actinomycin D (60 ng/ml), a DNA-intercalator, human primary fibroblast (MRC5), primary mouse embryonic fibroblasts (MEF) from p53−/− and p53+/+ littermate mice and human tumour cell lines expressing or not expressing wt p53. Actinomycin D used at 60 ng/ml does not prevent RNA polymerase II activity but activates strongly p53 (Blattner et al., 1999). Proteins were extracted after treatment and levels analysed by Western blot Waf and p53 protein levels were used as an indication of p53 activation. Scotin protein is clearly accumulated after UV irradiation or Actinomycin D treatment in mouse p53+/+ MEF, human primary fibroblast and human tumour cells expressing wt p53 (FIG. 7a, b) but not in mouse p53−/− MEF or human tumour cell lines devoid of p53 expression. Scotin induction is strictly p53-dependent since p53−/− MEF or Saos-2 that undergo apoptosis after UV radiation or actinomycin D treatment, respectively, do not induce Scotin. This suggests that Scotin can be induced in response to various stresses but only in cells expressing functional wt p53.

To determine whether p53 expression is sufficient to induce Scotin expression, we used two stable inducible cell lines. These are human p53 null cells (H1299 and Saos-2) that contain stably integrated wild type p53 cDNA whose expression is controlled by the tetracycline inducible system. In both cell lines, Scotin was induced following the activation of wt p53 while no Scotin induction could be detected in the control mutant p53his169-tetracycline inducible Saos-2 cells (FIG. 7c).

Altogether, these results show that induction of human and mouse Scotin protein requires wtp53 activation. Moreover, wild type p53 expression is sufficient to induce Scotin expression in the absence of cellular stress.

Scotin Protein is Localised in the Endoplasmic Reticulum (ER)

To determine the sub-cellular localisation of the endogenous Scotin protein after DNA damage, we exposed to UV (20 J/m2) p53+/+ MEF cells which express wtp53. Twenty-four hours after treatment, cells were fixed and stained by mouse anti-Scotin antibody respectively. A bright ring around the nucleus was observed in cells treated but not in control untreated cells (FIG. 8a, b). Similar localisation was observed in NIH3T3 fibroblast.

To determine the sub-cellular localisation of endogenous Scotin, we treated MCF-7 cells with Actinomycin D for 6 h. After fixation, cells were co-stained for Scotin and the gp96/GRP94 protein. gp96/GRP94 is a chaperone protein predominantly expressed in the ER (Koch et al., 1986; Li and Srivastava, 1993). As shown by confocal microscopy (FIG. 8c, d, e), gp96/GRP94 and Scotin were colocalised. This confirms that endogenous mouse or human Scotin proteins are localised in the endoplasmic reticulum (ER) and /or the nuclear membrane after cellular stress.

As we planned to use transfection method to study Scotin biological activity, it was essential to determine whether the sub-cellular localisation of ectopic Scotin protein was identical. Human H1299 lung carcinoma cells were transiently transfected with a mouse Scotin expression vector. To mimic physiological expression levels as closely as possible, we used Scotin expression vectors driven either by the SV40 promoter or the weak minimal major late promoter from adenovirus (mAdMLP). Twenty-four hours after transfection, cells were fixed and indirectly stained with the anti-mouse Scotin antibody. A bright ring around the nucleus was observed in H1299 cells transfected with AdScotin vector (FIG. 8e). We observed the same staining pattern after transient transfection in Saos-2, U2OS and NIH3T3 cell lines (data not shown). Moreover, as judged by immunostaining, transfection of AdScotin plasmid did not give rise, at the cellular level, to a strong overexpression of Scotin but to a level close to the endogenous Scotin expressed after cellular stress in MEF or MCF-7 cells. Transfection of 10 μg of SVScotin vector gave rise to a strong overexpression of Scotin in some H1299 cells revealing the characteristic staining pattern of the ER and of the nuclear membrane (FIG. 8f). This indicates that Scotin is expressed at the same localisation after transfection as endogenous Scotin protein.

To determine whether Scotin could be expressed in other cellular compartment, we overexpressed Scotin after transfection and analysed Scotin localisation by confocal microscopy after co-immunostaining with diverse organelle markers. As several antibodies for the markers were rabbit polyclonal antibodies, we fused a FLAG peptide at the C-terminus of the full mouse Scotin ORF. H1299 cells were transiently transfected with Scotin-Flag expression vectors driven by SV40 or mAdMLP promoters. In co-immunostaining, anti-Flag and anti-Scotin antibodies stained exactly the same cells at the same sub-cellular localisation. Scotin sub-cellular localisation was not affected by the Flag fusion (data not shown).

H1299 cells transfected with SVScotin-Flag plasmids were fixed 24 h, 48 h and 66 h after transfection and co-stained with the mouse monoclonal anti-Flag (M2) antibody followed by FITC-conjugated anti-mouse antibody and the rabbit polyclonal anti-gp96/GRP94 antibody followed by Texas-Red conjugated anti-rabbit antibody. As shown by confocal microscopy (FIG. 8h,i,j), gp96/GRP94 and Scotin were colocalised 24 h after transfection. The Scotin localisation was unchanged at 48 h and 66 h after transfection (data not shown). The same results were obtained after co-localisation with Calnexin, another protein exclusively expressed in the ER (data not shown). We did not detect Scotin in the cytoplasmic membrane even 66 h after transfection.

To determine whether Scotin could be expressed in the Golgi apparatus, we transfected H1299 cells with AdScotin-Flag vector. Cells were fixed 24 h, 48 h and 66 h after transfection and stained with anti-Flag and anti-TGN46 antibodies (Prescott et al., 1997). TGN46 protein is exclusively expressed in the Golgi apparatus (Prescott et al., 1997). By confocal microscopy, we did not observe a co-localisation of TGN46 and Scotin proteins 24 h, 48 h or 66 h after transfection (FIG. 8k, l, m).

To determine whether Scotin could be expressed in mitochondria, we transfected H1299 cells with SVScotin-Flag vector. Twenty-four hours after transfection, cells were incubated for 30 min with Red Mitotracker dye, which is incorporated specifically in mitochondria. Cells were then fixed and immunostained with anti-Flag antibody. By confocal microscopy, we observed that Scotin staining pattern is different from mitochondria staining pattern (compared FIG. 8n to FIG. 8o). On the merge FIG. 8p, Scotin staining did not co-localised exactly with mitochondria staining.

Taken together, these results indicates that Scotin protein is mainly located in the ER and can be located in the nuclear envelope in cells overexpressing Scotin after transfection. However, we cannot rule out the possibility that a small fraction of Scotin proteins can be localised in other cellular membranes.

Scotin can Promote Apoptosis Independently of p53

We noticed that induction of Scotin protein was coincident with cell death in wt p53 expressing cell lines (MRC5, MEF P53+/+, NIH3T3, MCF7 and U2OS) treated by UV or Actinomycin D suggesting that Scotin expression was associated with cell death.

To determine whether Scotin can be involved in cell death independently of p53, we sought to transfect the Scotin expression vectors into H1299 or Saos-2 cells that do not express p53. However, Scotin is an ER located protein and the ER can trigger cell signals leading to apoptosis in response to stresses that impair its functions such as protein overexpression after transfection or misfolded protein, hypoxia, inhibition of glycosylation and disruption of the ER calcium store (for review, Kaufman 1999). Therefore, we made three different Scotin mutants to determine whether Scotin protein expressed after transfection was cytotoxic due to an intrinsic activity (FIG. 9.1). The first mutant was generated by in frame deletion of the cysteine rich domain and subcloned in mAdMLP vector (AdΔCys). The second mutant had an in frame deletion of the entire N-terminus and was subcloned in mAdMLP expression vector (AdΔN). The third mutant was generated by deletion of the proline/tyrosine domain in the carboxyl end and subcloned into an SV40 expression vector (SvΔpro). All mutant proteins were fused at the C-terminal end to the Flag peptide. After transfection in H1299 cells and stag by anti-Flag antibody, the AdΔCys Scotin was localised in the ER in a similar pattern to AdScotin (FIG. 9.2c, 9.2d versus FIG. 9.2a). The AdΔN Scotin was mostly localised in the ER and the nuclear envelope in a similar pattern to SVScotin (FIG. 9.2e, 9.2f versus FIG. 9.2b). The SVΔpro Scotin lost its ER localisation and was expressed throughout the cytoplasm (FIG. 9.2g, 9.2h versus 9.2b).

We performed a clonogenic assay after co-transfection of AdScotin or SVScotin or AdΔCys or AdΔN or SVΔpro with a vector expressing the neomycin resistance gene in the cell lines H1299 and Saos-2 devoid of p53 to determine whether Scotin could reduce cell viability independently of p53. No clone stably overexpressing wild type Scotin could be obtained in any cell lines after selection by G418. However, we were able to obtain cells overexpressing in stable manner AdΔCys or AdΔN or SVΔpro Scotin mutants (data not shown). This suggested that wild type Scotin protein might prevent colony outgrowth and that Scotin mutants might have lost this activity. However, we could not rule out that the absence of clone overexpressing wild-type Scotin was due to a poor transfection efficiency of the AdScotin or SVScotin vectors.

To rule out this possibility, we performed a rapid and easy test based on residual luciferase activity after transient transfection (FIG. 9.3). H1299 cells were co-transfected with luciferase (Adluc), Renilla luciferase (SVRenilla) expression plasmids and wt or mutant Scotin expression vectors. To rule out variations in transfection efficiency, cells were transfected in duplicate with the same transfection mix and harvested 18 h, 28 h, 42 h and 52 h after transfection. Luciferase and Renilla luciferase activities were analysed independently by using the dual luciferase reporter kit from Primage. As negative controls we co-transfected the luciferase expression plasmids with pAdCAT plasmid, encoding the Chloramphenicol Acetyl Transferase enzyme, or the empty expression vector pSV40. As a positive control, we co-transfected the luciferase expression plasmids with wt p53 expression vector driven by the SV40 promoter. We estimated the reduction of cell viability by the relative residual luciferase activity calculated as the average of the residual luciferase activities at 42 h and 52 h divided by the average of the residual luciferase activities at 18 h and 28 h after transfection. In case of inhibition of cell viability, the relative residual luciferase activity is expected to be inferior or equal to 1. The renilla luciferase and firefly luciferase relative activities were calculated separately and presented in FIG. 9.3. As expected, co-transfection of AdCAT or pSV40 empty plasmids with Adluc and SVRenilla resulted in relative residual luciferase activities close to 2, which is consistent with the absence of cytotoxic activity carried by those plasmids. Co-transfection of p53 expression vector with Adluc and SVRenilla resulted in relative residual luciferase activities inferior to 1, which is consistent with the transexpression and the pro-apoptotic activities of p53 (Yonish-Rouach et al., 1991; Haupt et al., 1995). In cells co-transfected with AdScotin or SVScotin, both relative residual luciferase activities were close to 1. This suggests that the decreases of both luciferase activities were due to a reduction of cell viability rather than a specific activity of Scotin on the promoters or on the enzymatic activities of the luciferase proteins. In cells co-transfected with AdΔN or AdΔCys or SVΔpro Scotin mutant expression vectors, both relative residual luciferase activities were close to 2 suggesting that the Scotin mutants have lost the cytotoxic activity. These results suggested that Scotin protein could reduce cell viability independently of p53. Moreover, as AdΔCys and AdΔN Scotin mutants that are expressed in the ER have lost the wt Scotin activity, it suggested that Scotin biological activity was due to an intrinsic activity localised in the cysteine domain and not simply due to overexpression after transfection of an ER located protein. Furthermore, as the SVΔpro Scotin mutant, which is not localised in the ER, has lost the cytotoxic activity, it suggested that the proline rich region and/or the localisation of Scotin in the ER is essential to Scotin activity.

To determine if the Scotin-mediated decrease of relative residual luciferase activity was due to cell death, we performed a flow cytometry analysis as previously described to quantify apoptosis induced by p53 (Yonish-Rouach et al., 1994; Haupt et al., 1995). H1299 cells were transiently transfected with mouse wt or mutant Scotin-Flag expression vectors. Cells were collected 48 h after transfection, fixed and indirectly stained by anti-Flag antibody. DNA was stained with propidium iodide. As a positive control, cells were transfected in parallel with a wt p53 expression vector and stained for p53 48 h after transfection. We used a flow cytometry analysis to determine the DNA content of Scotin or p53 transfected cells. The profile of a representative experiment in H1299 cells transfected by SVScotin is shown in FIG. 10.1. We employed a three-parameter analysis to design the appropriate gating to separate the transfected cell population from the non-transfected cell population and cells debris. This approach ensures that the sub-G1 population analysed subsequently is composed of apoptotic cells and not simply debris.

The results of at least three independent experiments are summarised in FIG. 11 and Table 1.

TABLE 1
Scotin mediated-apoptosis is p53-independent
but caspase-dependent

| | % sub-G1 | exp |
|---|---|---|
| non-transfected | 1.5 | 1064434223242 |
| SVScotin 2 μg | 7.4 | |
| SVScotin 10 μg | 13.1 | |
| AdScotin 5 μg | 10.3 | |
| AdΔCys 5 μg | 4.6 | |
| AdΔN 5 μg | 4.3 | |
| AdScotin 5 μG + AdCat 5 μg | 10.7 | |
| AdScotin 5 μg + AdΔN 5 μg | 6 | |
| Adscotin 5 μg + AdΔCys 5 μg | 5.7 | |
| AdScotin 5 μg + capase inhibitor cocktail | 4.15 | |
| Svp53 2 μg | 16.5 | |
| Svp53 1 μg + AdΔCys 5 μg | 14.2 | |

H1299 cells transfected with different expression vectors were harvested 48 h after transfection. The DNA content of each transfected population was determined by three parameters flow cytometry analysis as described FIG. 10. The percentage of sub-G1 DNA content represents percentage of apoptotic cells. Caspase inhibitor cocktail (10 μM) was added 4 h before transfection. The average of at least two independent transfections is presented. The number of experiments realised is indicated (exp).

Transient transfection of SVp53 expression vector caused 16% of transfected cells to have a sub-G1 DNA content, which is indicative of cell death, in agreement with earlier reports (Yonish-Rouach et al., 1994; Haupt et al., 1995) (FIG. 11a: 2, 3). The mouse Scotin expressing cells transfected with an increasing concentration of plasmid SVScotin-Flag (FIG. 11a: 4, 5, 6) or AdScotin-Flag (FIG. 11a: 7, 8) exhibited in a concentration-dependent manner a significantly higher fraction of cells with sub-G1 DNA content (15% and 12% respectively) compared to the counterpart non-transfected cells (1.5%) demonstrating that Scotin expression is cytotoxic. In contrast, the fraction of cells with sub-G1 DNA content expressing AdΔCys or AdΔN Scotin mutant (FIG. 11a: 9, 10) represented only 4.5% of the total AdΔCys or AdΔN Scotin mutant transfected cells. Although this percentage is significantly higher than the corresponding fraction in non-transfected cells, it is significantly lower than the corresponding fraction in AdScotin-Flag expressing cells. This confirms that both Scotin mutant proteins deleted of the cysteine domain have lost most of the cytotoxic activity.

Importantly, co-transfection of AdΔCys or AdΔN Scotin mutant with AdScotin-Flag expression vector reduced the fraction of Scotin expressing cells with sub-G1 DNA content by 50% (FIG. 11a: 11, 12, 13) although the expression of wt Scotin was not reduced by AdΔN Scotin mutant co-expression (FIG. 11b), suggesting that Scotin mutant proteins can act as dominant negatives over wt Scotin protein. This confirms that Scotin-mediated cell death is not simply due to the expression of Scotin protein in the ER but specifically requires an intrinsic activity contained in the cysteine rich domain. Interestingly, when H1299 cells were incubated with a cocktail of caspase inhibitors prior transfection with AdScotin-Flag or SV Scotin-Flag, Scotin-mediated cell death can be inhibited suggesting that Scotin induces apoptosis in a caspase-dependent manner (FIG. 11a: 14, 15).

In order to confirm that the cell death induced by Scotin was apoptosis, we transiently transfected the Scotin expression vectors into H1299 or Saos-2 cells seeded on slides. Cells were fixed 40 h after transfection. We performed a TUNEL assay to stain nuclei presenting DNA breaks and cells were stained by indirect fluorescence (Texas-Red) with polyclonal anti-Scotin antibody. DNA was co-stained by DAPI to correlate DNA condensation and nucleus fragmentation with TUNEL positive cells indicating the cell death is by apoptosis. As shown FIG. 10.2, TUNEL positive cells presented nuclei fragmentation or condensed DNA and exhibited a strong staining for Scotin confirming that cells with a sub-G1 DNA content observed in the flow cytometry analysis corresponded to cells in apoptosis.

Altogether these results show that the ER-located protein Scotin can induce apoptosis in a caspase dependent manner but independently of p53. Moreover, Scotin-mediated apoptosis is due to an intrinsic pro-apoptotic activity localised in the cysteine rich domain of Scotin protein and not simply due to overexpression after transfection of an ER located protein. Therefore, Scotin protein might play a role in p53-mediated apoptosis.

Scotin Protein is Required to Induce Apoptosis in Response to ER Stress

To assess the role of Scotin in apoptosis under physiological conditions, NIH3T3 cells were transfected in a stable manner with an antisense Scotin expression vector (see Experimental Procedure above). As a control, NIH3T3 cells were transfected in a stable manner with pcDNA3 expression vector expressing a non-coding sequence not related to Scotin or other known genes. Control and Scotin antisense expressing cells were exposed for 24 h or 42 h to actinomycin D (60 ng/ml). Proteins were extracted after treatment and analysed by Western blot for Scotin expression (FIG. 12.1). Scotin basal level was detectable and well induced after treatment in control antisense expressing cells. Scotin was barely detectable in Scotin antisense expressing cells despite a strong activation of p53 after actinomycin D treatment demonstrating that Scotin antisense expression vector inhibited endogenous Scotin expression strongly.

To determine if Scotin plays a role in the p53-mediated apoptosis induced by DNA-damage agents, we treated control and Scotin antisense cells with UV or doxorubicin (FIG. 12.3). Cells were treated with different doses of UV and the number of cells alive 24 h after treatment was determined by trypan blue analysis (FIG. 12.2). Scotin antisense cells are more resistant to apoptosis 24 h after UV treatment than control antisense cells, particularly after a dose of 15 J/m2.

Cells were treated with different doses of doxorubicin for 24 h and were allowed to recover for 24 h after treatment. Cell survival was estimated by giemsa staining. As reported on FIG. 12.3, Scotin antisense cells are more resistant to cell death induced by doxorubicin than control antisense cells. Altogether, this indicates that Scotin is required for p53-mediated apoptosis induced by DNA-damage.

Recent studies suggest that in response to ER stress, the ER can trigger cell signals inducing apoptosis (Wang et al., 1998; Zinszner et al., 1998; Kaufman, 1999). As Scotin is located in the ER, it is postulated whether Scotin could be involved in the apoptosis induced by ER stress. The list of conditions known to trigger the ER stress response includes treatment of cells with thapsigargin, which interferes with calcium flux across the ER membrane, or tunicamycin, an inhibitor of N-linked glycosylation, or reducing agents, or deprivation of nutrients such as glucose, amino acids, or hypoxia. Normal mouse fibroblasts undergo a massive apoptosis after treatment with tunicamycin or thapsigargin (Zinszner et al., 1998). NIH3T3, p53−/−, Scotin antisense and control antisense expressing fibroblasts were treated with different doses of thapsigargin or tunicamycin or FCCP, a protonophore inducing mitochondrial stress. Cell survival was estimated by giemsa staining (FIG. 12.3). Scotin antisense cells were more resistant than control antisense cells to cell death induced by tunicamycin or thapsigargin but not to FCCP indicating that Scotin is specifically required for cell death induced by ER-stress but has no effect on mitochondrial stress. Cell death induced by tunicamycin or thapsigargin was apoptosis as shown on FIG. 12.4.

These results demonstrate that Scotin is a pro-apoptotic protein under physiological conditions. Scotin expression is required to induce apoptosis in response to alterations of the endoplasmic reticulum functions and DNA-damage.

As p53−/− fibroblasts are resistant to apoptosis induced by thapsigargin or tunicamycin treatment, it suggests that ER stress-mediated apoptosis is p53 dependent. In agreement, we noted that 40 h after treatment with thapsigargin, p53 is accumulated in NIH3T3 and control antisense expressing fibroblasts indicating that ER stresses can activate p53 (data not shown). Altogether, results show that p53 activated by ER stress induces Scotin, which triggers apoptosis in a caspase dependent manner.

### Discussion

Scotin Gene is Induced in a p53 Dependent Manner

Few pro-apoptotic genes directly induced by p53 have been described. This is probably due to the use of cellular p53 models, which being derived from tumours or immortalised primary cells are likely to have lost some pro-apoptotic gene expressions as an adaptation to in vitro culture. It has been shown that thymus and spleen cells undergo a massive p53-dependent apoptosis after ionising radiation in normal mice but not in p53 nullizygote mice (Lowe et al., 1993; Clarke et al., 1993 Midgley et al., 1995). This animal model can thus be used to identify new genes involved in p53-mediated apoptosis induced by irradiation. In the present study, we report the identification and characterisation of a novel gene, named Scotin, which is induced, in vivo, after ionising radiation in a p53-dependent manner.

Two rabbit polyclonal antibodies were raised from two peptides corresponding to the C-terminal end of human and mouse Scotin respectively. We showed that human and mouse Scotin proteins are induced in response to cellular stress in a p53-dependent manner. However, Scotin protein is constitutively expressed at a basal level in a p53 independent manner. By using a tetracycline-inducible p53 system we showed that the p53-mediated Scotin induction does not require cellular stresses suggesting that wild type p53 expression is necessary and sufficient to induce Scotin expression. The first intron and 650 bp of the promoter containing the transcription initiation site have been cloned, sequenced and studied in luciferase reporter assay. The first intron or the promoter region are not responsive to p53 despite the presence of a potential p53-binding site (2 motifs PuPuPuCA/TA/TGPyPyPy separated by 1 bp) in the promoter region. We are currently isolating a longer region of the mouse Scotin promoter to determine whether the Scotin gene is directly transactivated by p53.

Scotin Gene is Conserved Between Mouse and Human and Belongs to a Gene Family

Mouse Scotin cDNA was completed by RACE PCR and used in a computer analysis of EST sequences (dbEST database) contained in GenBank to identify mouse and human

Scotin homologous cDNA. We identified two sets of mouse EST sequences homologous to mouse Scotin cDNA, one identical to Scotin cDNA and one with a different 5′end. We also identified two sets of EST sequences in human, homologous to the two sets previously identified in mouse, suggesting that the Scotin gene belongs to a conserved family of genes.

The Scotin protein sequence and structure is well conserved between human and mouse. The proline/tyrosine domain contains several protein-protein interaction motifs whose some can be regulated by tyrosine phosphorylation; 2 SH2 binding motifs (p-Yxxψ), 1 PTB binding motif (NPxY), 2 WW binding motifs (PPxY) and 5 SH3 binding motifs (PxxP). Since the motifs are conserved, the carboxyl-end of Scotin might be phosphorylated on tyrosine. Scotin might be a transmembrane receptor, which, after interaction with a ligand at its N-terminus, would induce a cell signal transduction in the cytoplasm through its carboxyl-terminus.

The Scotin-related protein is conserved between mouse and human but diverges from Scotin protein in the N-terminus and in the terminal part of the carboxyl half. Further study will determine if this Scotin-related protein is involved in apoptosis.

After completion of human Scotin cDNA by RACE PCR, we analysed the dbEST database to determine if EST sequences corresponding to human Scotin are potentially expressed in tumours, normal tissues and during development. We identified 104 human Scotin EST sequences identical (99%) over 200 to 400 bp to the human Scotin 3′end cDNA. Scotin was found to be expressed in a wide range of human foetal tissue (heart, lung, liver, placenta), normal tissue (bone, pineal gland, thymus, spleen, prostate, bone marrow, ovary, breast, testis, liver) and tumours of various origins (uterus, colon, brain, prostate, ovary, leukaemia, kidney, sarcoma, pancreas, stomach, cervix) indicating that Scotin expression is not restricted to spleen and thymus. Moreover, as Scotin was found expressed in a wide range of human cancers, Scotin protein may constitute an interesting target for future cancer diagostics and therapies. However, further studies are necessary to confirm this computer analysis. We are currently studying the Scotin protein expression profile in adult and foetal tissues and characterising Scotin gene status in cell lines and tumours.

Scotin is an ER-located Protein

Primary and secondary structures predict that Scotin protein is a transmembrane receptor suggesting that Scotin can then be involved in cell signalling. It was therefore surprising to find Scotin located in the ER after cellular stress or ectopic transfection. To determine if Scotin could be expressed in other subcellular compartments, we strongly overexpressed Scotin by transfection. Scotin was not detected by immunostaining, even 66 h after transfection, in the Golgi apparatus or cytoplasmic membrane but it was present in the ER and the nuclear envelope, suggesting that the biochemical activity of Scotin could depend on the ER functions.

The Scotin mutant deleted of the first 22 amino acids and the cysteine domain (AdΔN) is located in the nuclear envelope and the ER while the mutant deleted only of the cysteine domain (AdΔCys) or wt Scotin (AdScotin) are only located in the ER. It suggests that the first 22 amino acids are required to the localisation of Scotin in the ER. The Scotin mutant deleted of the carboxyl end produced by SV40 promoter (SVΔpro) is not located in the ER but throughout the cytoplasm although it contains the first 22 amino acids. However, wt Scotin protein also produced by SV40 promoter (SVScotin) is well localised in the ER and the nuclear membrane probably because of the high expression level. This suggests that the carboxyl half of Scotin is absolutely required for the localisation in the ER and the nuclear membrane. The localisation of Scotin in the ER requires the carboxyl half and the first 22 amino acids, which might constitute a signal sequence.

Scotin can Promote Apoptosis Independently of p53

We demonstrated by clonogenic assay, residual luciferase activity assay, three-parameter flow cytometry analysis and TUNEL assay that transfection of Scotin expression vectors driven by weak promoters can induce apoptosis in different cell lines independently of p53 but in a caspase-dependent manner. To determine if Scotin-mediated apoptosis was due to a specific activity of Scotin protein, we generated different Scotin mutants. We showed that both Scotin mutants deleted of the cysteine rich domain have reduced pro-apoptotic activity although expressed in the ER. This indicates that Scotin mediated-apoptosis is due to an intrinsic activity of the Scotin protein. Moreover, overexpression of such Scotin mutants can act as dominant negative over wt Scotin suggesting that Scotin mutants deleted of the cysteine domain compete with wt Scotin for a same ligand.

Scotin Promotes Apoptosis Caused by Impairment of the ER Functions

Evidence is emerging that the ER plays a major role in apoptosis. As a protein-folding compartment, the ER is exquisitely sensitive to alterations in homeostasis that disrupt ER functions. ER stresses include ER calcium store depletion, inhibition of glycosylation, reduction of disulfide bond, expression of mutant protein or protein subunits, overexpression of wild-type protein, expression of viral proteins, TNFα treatment, hypoxia, (for review, Kaufman, 1999). Sustained elevation of cytosolic [Ca²⁺] can induce cell death by apoptosis (McConkey and Orrenius, 1997; Nicotera and Orrenius, 1998). The release of calcium from the ER upon pro-apoptotic signalling or after thapsigargin treatment, triggers the opening of the calcium-sensitive mitochondrial permeability transition pore (PTP) allowing the release of cytochrome c from the mitochondria to the cytosol (Ichas et al., 1997; Ichas and Mazat, 1998; Szalai et al., 1999). The cytosolic cytochrome c binds Apaf-1 and procaspase-9 leading to caspase-9 activation, which then processes and activates other caspases to orchestrate the programmed cell death (Li et al., 1997), (for review see Green and Reed, 1998). Moreover, calcium-mediated apoptosis can be inhibited by Bcl-2 expression that can maintain Ca²⁺ homeostasis within the ER (Lam et al., 1994; Marin et al., 1996; He et al., 1997; Kuo et al., 1998).

Recent studies have shown that the ER can also generate cell signals in response to ER stress that lead to apoptosis induction via activation of the transcription factor CHOP (GADD153) (Zinszner et al., 1998; Kaufman, 1999). Wang et al., (1998) have shown that overexpression after transfection of the ER-associated type 1 transmembrane protein kinase (Ire1), a sensor of ER stress, can activate CHOP expression. However, the mechanism of activation of apoptosis by Ire1 overexpression or in response to ER stress is still poorly understood.

As Scotin is an ER located protein that can promote apoptosis after transfection, we wondered whether Scotin can trigger apoptosis in response to ER stress caused by calcium release from the ER upon thapsigargin treatment or by inhibition of the N-glycosylation reaction following tunicamycin treatment. We inhibited endogenous Scotin expression in NIH3T3 cells by transfecting, in a stable manner, a mouse Scotin antisense expression vector. We showed that inhibition of Scotin protein expression in NIH3T3-derived Scotin antisense cells strongly increased resistance to apoptosis induced by thapsigargin or tunicamycin treatment in comparison to NIH3T3-derived control antisense cells. This demonstrates that Scotin is a pro-apoptotic protein under physiological stress and that Scotin is required to induce apoptosis in response to impairment of the ER functions. Scotin has therefore all the characterstics expected of a gene that can contribute to the p53-mediated apoptosis. It would be interesting to determine whether TNF or Fas or Bax-mediated apoptosis require Scotin expression and whether the anti-apoptotic protein Bcl2, which is also expressed in the ER, can inhibit Scotin-mediated apoptosis.

In conclusion, in response to cellular stress, p53 induces the Scotin gene whose gene product promotes apoptosis independently of p53 but in a caspase-dependent manner. Scotin is a pro-apoptotic transmembrane protein located in the ER, which is required to induce apoptosis in response to ER stress. The discovery of Scotin clarifies the role of the ER in apoptosis and indicates that impairment of the ER functions may trigger a cell signaling from the ER activating p53 that can be at the origin of the cell death by apoptosis. It brings to light the role of the endoplasmic reticulum stress signalling in p53-mediated apoptosis.

It is to be understood that the above is merely exemplary and is not to be construed as limiting in any way.

### Bibliography

Attardi, L. D., Lowe, S. W., Brugarolas, J., and Jacks, T. (1996). Transcriptional activation by p53, but not induction of the p21 gene, is essential for oncogene-mediated apoptosis. Embo J 15, 3693-701.

Barak, Y., Gottlieb, E., Juven-Gershon, T., and Oren, M. (1994). Regulation of mdm2 expression by p53: alternative promoters produce transcripts with nonidentical translation potential. Genes Dev 8, 1739-49.

Blattner, C., Sparks, A., and Lane, D. (1999). Transcription factor E2F-1 is upregulated in response to DNA damage in a manner analogous to that of p53. Mol Cell Biol 19, 3704-13.

Bourdon, J. C., Deguin-Chambon, V., Lelong, J. C., Dessen, P., May, P., Debuire, B., and May, E. (1997). Further characterisation of the p53 responsive element—identification of new candidate genes for trans-activation by p53. Oncogene 14, 85-94.

Brugarolas, J., Chandrasekaran, C., Gordon, J. I., Beach, D., Jacks, T., and Hannon, G. J. (1995). Radiation-induced cell cycle arrest compromised by p21 deficiency. Nature 377, 552-7.

Buckbinder, L., Talbott, R, Velasco-Miguel, S., Takenaka, I., Faha, B., Seizinger, B. R., and Kley, N. (1995). Induction of the growth inhibitor IGF-binding protein 3 by p53. Nature 377, 646-9.

Caelles, C., Helmberg, A., and Karin, M. (1994). p53-dependent apoptosis in the absence of transcriptional activation of p53-target genes [see comments]. Nature 370, 220-3.

Chan, T. A, Hermeking, H, Lengauer, C., Kinzler, K. W., and Vogelstein, B. (1999). 14-3-3 Sigma is required to prevent mitotic catastrophe after DNA damage [see comments]. Nature 401, 616-20.

Clarke, A. R, Purdie, C. A, Harrison, D. J., Morris, R G., Bird, C. C., Hooper, M. L., and Wyllie, A. H. (1993). Thymocyte apoptosis induced by p53-dependent and independent pathways [see comments]. Nature 362, 849-52.

Deng, C., Zhang, P., Harper, J. W., Elledge, S. J., and Leder, P. (1995). Mice lacking p21CIP1/WAF1 undergo normal development, but are defective in G1 checkpoint control. Cell 82, 675-84.

Donehower, L. A.,Harvey, M., Slagle, B. L., McArthur, M. J., Montgomery, C. A., Jr., Butel, J. S., and Bradley, A (1992). Mice deficient for p53 are developmentally normal but susceptible to spontaneous tumours. Nature 356, 215-21.

El-Deiry, W. S., Kern, S. E., Pietenpol, J. A., Kinzler, K. W., and Vogelstein, B. (1992). Definition of a consensus binding site for p53. Nat Genet 1, 45-9.

El-Deiry, W. S., Tokino, T., Velculescu, V. E., Levy, D. B., Parsons, R, Trent, J. M., Lin, D., Mercer, W. E., Kinzler, K. W., and Vogelstein, B. (1993). WAF1, a potential mediator of p53 tumor suppression. Cell 75, 817-25.

Funk, W. D., Pak, D. T., Karas, R. H., Wright, W. E., and Shay, J. W. (1992). A transcriptionally active DNA-binding site for human p53 protein complexes. Mol Cell Biol 12, 2866-71.

Gossen, M., Freundlieb, S., Bender, G., Muller, G., Hillen, W., and Bujard, H. (1995). Transcriptional activation by tetracyclines in mammalian cells. Science 268, 1766-9.

Gottlieb, T. M., and Oren, M. (1996). p53 in growth control and neoplasia. Biochimica Biophysica Acta 1287, 77-102.

Graeber, T. G., Peterson, J. F., Tsai, M., Monica, K, Fornace, A., Jr., and Giaccia, A. J. (1994). Hypoxia induces accumulation of p53 protein, but activation of a G1-phase checkpoint by low-oxygen conditions is independent of p53 status. Mol Cell Biol 14, 6264-77.

Green, D. R, and Reed, J. C. (1998). Mitochondria and apoptosis. Science 281, 1309-12.

Hall, P. A., McKee, P. H., Menage, H. D., Dover, R., and Lane, D. P. (1993). High levels of p53 protein in UV-irradiated normal human skin. Oncogene 8, 203-7.

Harlow E and Lane D. P. (1988). Antibodies, a laboratory manual, eds. (Cold Spring Harbor Laboratories).

Harper, J. W., Adami, G. R., Wei, N., Keyomarsi K., and Elledge, S. J. (1993). The p21 CdK-interacting protein Cip1 is a Potent inhibitor of G1 cyclin-dependent kinases. Cell 75, 805-816.

Haupt, Y., Rowan, S., Shaulian, E., Vousden, K. H., and Oren, M. (1995). Induction of apoptosis in HeLa cells by trans-activation-deficient p53. Genes Dev 9, 2170-83.

He, H., Lam, M., McCormick, T. S., and Distelhorst, C. W. (1997). Maintenance of calcium homeostasis in the endoplasmic reticulum by Bcl-2. J Cell Biol 138, 1219-28.

Hermeking, H., Lengauer, C., Polyak, K., He, T. C., Zhang, L., Thiagalingam, S., Kinzler, K. W., and Vogelstein, B. (1997). 14-3-3 sigma is a p53-regulated inhibitor of G2/M progression. Mol Cell 1, 3-11.

Hupp, T. R, Sparks, A., and Lane, D. P. (1995). Small peptides activate the latent sequence-specific DNA binding function of p53. Cell 83, 237-45.

Hollstein, M., Sidransky, D., Vogelstein, B., and Harris, C. C. (1991). p53 mutations in human cancers. Science 253, 49-53.

Ichas, F., Jouaville, L. S., and Mazat, J. P. (1997). Mitochondria are excitable organelles capable of generating and conveying electrical and calcium signals. Cell 89, 1145-53.

Ichas, F., and Mazat, J. P. (1998). From calcium signaling to cell death: two conformations for the mitochondrial permeability transition pore. Switching from low- to high-conductance state. Biochim Biophys Acta 1366, 33-50.

Kastan, M. B., Onyekwere, O., Sidransky, D., Vogelstein, B., and Craig, R. W. (1991). Participation of p53 protein in the cellular response to DNA damage. Cancer Res 51, 6304-11.

Kastan, M. B., Zhan, Q., el-Deiry, W. S., Carrier, F., Jacks, T., Walsh, W. V., Plunkett, B. S., Vogelstein, B., and Fornace, A., Jr. (1992). A mammalian cell cycle checkpoint pathway utilizing p53 and GADD45 is defective in ataxia-telangiectasia Cell 71, 587-97.

Kaufman, R. J.(1999). Stress signaling from the lumen of the endoplasmic reticulum: coordination of gene transcriptional and translational controls. Genes Dev 13, 1211-33.

Knudson, M. C., Tung, K. S. K., Tourtelotte, W. G., Brown, G. A. J., and Korsmeyer, S. J. (1995). Bax-deficient mice with lymphoid hyperplasia and male germ cell death Science 270, 96-98.

Ko, L. J., and Prives, C. (1996). p53: puzzle and paradigm. Genes Dev 10.

Koch, G., Smith, M., Macer, D., Webster, P., and Mortara, R. (1986). Endoplasmic reticulum contains a common, abundant calcium-binding glycoprotein, endoplasmin. J Cell Sci 86, 217-32.

Komarova, E. A., Chernov, M. V., Franks, R., Wang, K., Armin, G., Zelnick, C. R., Chin, D. M., Bacus, S. S., Stark, G. R., and Gudkov, A. V. (1997). Transgenic mice with p53-responsive lacZ: p53 activity varies dramatically during normal development and determines radiation and drug sensitivity in vivo. Embo J 16, 1391-400.

Kuo, T. H., Kim, H. R., Zhu, L., Yu, Y., Lin, H. M., and Tsang, W. (1998). Modulation of endoplasmic reticulum calcium pump by Bcl-2. Oncogene 17, 1903-10.

Lam, M., Dubyak, G., Chen, L., Nunez, G., Miesfeld, R. L., and Distelhorst, C. W. (1994). Evidence that BCL-2 represses apoptosis by regulating endoplasmic reticulum-associated Ca2+ fluxes. Proc Natl Acad Sci USA 91, 6569-73.

Lane, D. P. (1992). Cancer. p53, guardian of the genome [news; comment] [see comments]. Nature 358, 15-6.

Levine, A. J. (1997). p53, the cellular gatekeeper for growth and division. Cell 88, 323-31.

Li, P., Nijhawan, D., Budihardjo, I., Srinivasula, S. M., Ahmad, M., Alnemri, E. S., and Wang, X. (1997). Cytochrome c and dATP-dependent formation of Apaf-1/caspase-9 complex initiates an apoptotic protease cascade. Cell 91, 479-89.

Li, Z., and Srivastava, P. K. (1993). Tumor rejection antigen gp96/grp94 is an ATPase: implications for protein folding and antigen presentation. Embo J 12, 3143-51.

Liang, P., and Pardee, A. B. (1992). Differential display of eukaryotic messenger RNA by means of the polymerase chain reaction [see comments]. Science 257, 967-71.

Lowe, S. W., Schmitt, E. M., Smith, S. W., Osborne, B. A., and Jacks, T. (1993). p53 is required for radiation-induced apoptosis in mouse thymocytes [see comments]. Nature 362, 847-9.

Lu, X., and Lane, D. P. (1993). Differential induction of transcriptionally active p53 following UV or ionizing radiation: defects in chromosome instability syndromes? Cell 75, 765-78.

Lu, X, Burbidge, S. A., Griffin, S., and Smith, H. M. (1996). Discordance between accumulated p53 protein level and its transcriptional activity in response to u.v. radiation. Oncogene 13, 413-8.

Marin, M. C., Fernandez, A., Bick, R. J., Brisbay, S., Buja, L. M., Snuggs, M., McConkey, D. J., von Eschenbach, A. C., Keating, M. J., and McDonnell, T. J. (1996). Apoptosis suppression by bcl-2 is correlated with the regulation of nuclear and cytosolic Ca2+. Oncogene 12, 2259-66.

Mashimo, T., Watabe, M., Hirota, S., Hosobe, S., Miura, K., Tegtmeyer, P. J., Rinker-Shaeffer, C. W., and Watabe, K. (1998). The expression of the KAI1 gene, a tumor metastasis suppressor, is directly activated by p53. Proc Natl Acad Sci USA 95, 11307-11.

May, E., Mouriesse, H., May-Levin, F., Contesso, G., and Delarue, J. C. (1989). A new approach allowing an early prognosis in breast cancer: the ratio of estrogen receptor (ER) ligand binding activity to the ER-specific mRNA level. Oncogene 4, 1037-42.

McConkey, D. J., and Orrenius, S. (1997). The role of calcium in the regulation of apoptosis. Biochem Biophys Res Commun 239, 357-66.

Midgley, C. A., Fisher, C. J., Bartek, J., Vojtesek, B., Lane, D., and Barnes, D. M. (1992). Analysis of p53 expression inhuman tumours: an antibody raised against human p53 expressed in Escherichia coli. J Cell Sci 101, 183-9.

Midgley, C. A. Owens, B., Briscoe, C. V., Thomas, D. B., Lane, D. P., and Hall, P. A. (1995). Coupling between gamma irradiation, p53 induction and the apoptotic response depends upon cell type in vivo. J Cell Sci 108, 1843-8.

Miyashita, T., and Reed, J. C. (1995). Tumor suppressor p53 is a direct transcriptional activator of the human bax gene. Cell 80, 293-9.

Munsch, D., Watanabe-Fukunaga, R., Bourdon, J. C., Nagata, S., May, E., Yonish-Rouach, E., and Reisdorf, P. (2000). Human and Mouse Fas (APO-1/CD95) Death Receptor Genes Each Contain a p53-responsive Element That Is Activated by p⁵³ Mutants Unable to Induce Apoptosis. J Biol Chem 275, 3867-3872.

Nicotera, P., and Orrenius, S. (1998). The role of calcium in apoptosis. Cell Calcium 23, 173-80.

Nylander, K., Bourdon, J. C., Bray, S. E., Gibbs, N. K., Kay, R., Hart, I., and Hall, P. A. (2000). Transcriptional activation of tyrosinase and TRP-1 by p33 links UV irradiation to the protective tanning response [In Process Citation]. J Pathol 190, 39-46.

Okamoto, K., and Beach, D. (1994). Cyclin G is a transcriptional target of the p53 tumor suppressor protein. Embo J 13, 4816-22.

Oren, M. (1999). Regulation of the p53 tumor suppressor protein. J Biol Chem 274, 36031-4.

Prescott, A. R., Lucocq, J. M., James, J., Lister, J. M., and Ponnambalam, S. (1997). Distinct compartmentalization of TGN46 and beta 1,4-galactosyltransferase in HeLa cells. Eur J Cell Biol 72, 238-46.

Shaulsky, G., Goldfinger, N., Peled, A., and Rotter, V. (1991). Involvement of wild-type p53 in pre-B-cell differentiation in vitro. Proc Natl Acad Sci USA 88, 8982-6.

Stephen, C. W., Helminen, P., and Lane, D. P. (1995). Characterisation of epitopes on human p53 using phage-displayed peptide libraries: insights into antibody-peptide interactions. J Mol Biol 248, 58-78.

Szalai, G., Krishnamurthy, R., and Hajnoczcy, G. (1999). Apoptosis driven by IP(3)-linked mitochondrial calcium signals. Embo J 18, 6349-61.

Tokino, T., Thiagalingam, S., el-Deiry, W. S., Waldman, T., Kinzler, K. W., and Vogelstein, B. (1994). p53 tagged sites from human genomic DNA. Hum Mol Genet 3, 1537-42.

Utrera, R., Collavin, L., Lazarevic, D., Delia, D., and Schneider, C. (1998). A novel p53-inducible gene coding for a microtubule-localized protein with G2-phase-specific expression [In Process Citation]. Embo J 17, 5015-25.

Velasco-Miguel, S., Buckbinder, L., Jean, P., Gelbert, L., Talbott, R., Laidlaw, J., Seizinger, B., and Kley, N. (1999). PA26, a novel target of the p53 tumor suppressor and member of the GADD family of DNA damage and growth arrest inducible genes. Oncogene 18, 127-37.

Wang, X. Z., Harding, H. P., Zhang, Y., Jolicoeur, E. M., Kuroda, M., and Ron, D. (1998). Cloning of mammalian Ire1 reveals diversity in the ER stress responses. Embo J 17, 5708-17.

Waseem, N. H., and Lane, D. P. (1990). Monoclonal antibody analysis of the proliferating cell nuclear antigen (PCNA). Structural conservation and the detection of a nucleolar form J Cell Sci 96, 121-9.

Wu, X., Bayle, J. H., Olson, D., and Levine, A. J. (1993). The p53-mdm-2 autoregulatory feedback loop. Genes Dev 7, 1126-32.

Xiong, Y., Hannon, G. J., Zhang, H., Casso, D., Kobayashi, R., and Beach, D. (1993). p21 is a universal inhibitor of cyclin kinases [see comments]. Nature 366, 701-4.

Yin, C., Knudson, C. M., Korsmeyer, S. J., and Van Dyke, T. (1997). Bax suppresses tumorigenesis and stimulates apoptosis in vivo. Nature 385, 637-40.

Yonish-Rouach, E., Borde, J., Gotteland, M., Mishal, Z., Viron, A, and May, E. (1994). Induction of apoptosis by transiently transfected metabolically stable wt p53 in transformed cell lines. Cell Death Diff 1, 39-47.

Yonish-Rouach, E., Deguin, V., Zaitchouk, T., Breugnot, C., Mishal, Z., Jenkins, J. R., and May, E. (1996). Transcriptional activation plays a role in the induction of apoptosis by transiently transfected wild-type p53. Oncogene 11, 2197-205.

Yonish-Rouach, E., Resnitzky, D., Lotem, J.; Sachs, L., Kimchi, A., and Oren, M. (1991). Wild-type p53 induces apoptosis of myeloid leukaemic cells that is inhibited by interleukin-6. Nature 352, 345-7.

Zauberman, A., Lupo, A., and Oren, M. (1995). Identification of p53 target genes through immune selection of genomic DNA: the cyclin G gene contains two distinct p53 binding sites. Oncogene 10, 2361-6.

Zhan, Q., Carrier, F., and Fornace, A., Jr. (1993). Induction of cellular p53 activity by DNA-damaging agents and growth arrest [published erratum appears in Mol Cell Biol 1993 September; 13(9):5928]. Mol Cell Biol 13, 4242-50.

Zhao, S., Ooi, S. L., Yang, F. C., and Pardee, A. B. (1996). Three methods for identification of true positive cloned cDNA fragment in differential display. Biotechniques 20, 400-4.

Zinszner, H., Kuroda, M., Wang, X., Batchvarova, N., Lightfoot, R. T., Remotti, H., Stevens, J. L., and Ron, D. (1998). CHOP is implicated in programmed cell death in response to impaired function of the endoplasmic reticulum. Genes Dev 12, 982-95.

What is claimed is:

1. An isolated nucleotide sequence encoding a p53-inducible protein as shown in FIGS. 2, 3, 13, 14, 15, 16, 17, 18 or 19, derivative or fragment thereof or species specific homologue thereof.

2. An isolated nucleotide sequence according to claim 1, wherein said nucleotide sequence is from a mouse or a human.

3. An isolated nucleotide sequence which is complementary to the one which hybridises under stringent conditions with the nucleotide sequences of claim 1.

4. An isolated nucleotide sequence, wherein said nucleotide sequences have 75% identity, or above with the nucleotide sequences of claim 1.

5. An isolated nucleotide sequence complementary to the sequences of any preceeding claim.

6. An isolated nucleotide sequence according to any preceding claim, wherein said nucleotide sequence is for use in micro arrays, DNA arrays or DNA chips.

7. An isolated nucleotide sequence according to claim 6, wherein said nucleotide sequences are used to determine p53 activity and/or p53 responsiveness to cancer drug therapy from a biopsy.

8. An expression cassette comprising a promoter operably linked to any one of the nucleotide sequences of any one of claims 1 to 4.

9. A nucleotide sequence comprising a transcriptional regulatory sequence, and a sequence under the transcriptional control thereof which comprises an nucleotide sequence anti-sense to the nucleotide sequence of any one of the sequences of FIGS. 2, 3, 13, 14, 15, 16, 17, 18 or 19, derivative or fragment thereof or species specific homologue thereof.

10. A nucleotide sequence according to claim 9, wherein the length of said anti-sense sequence is 20 nucleotides in length up to the length of the mRNA molecule produced by the cell.

11. A nucleotide sequence according to claim 10, wherein said length is from 50 to 1500 nucleotides in length.

12. A pharmaceutical formulation comprising a polynucleotide fragment comprising the nucleotide sequence of any preceding claim, and a pharmacologically acceptable carrier.

13. A polypeptide as shown in FIGS. 4, 5, 20, 21, 22, 23 or 25, functionally active fragments, derivatives or homologues thereof.

14. A polypeptide which comprises the polypeptide of claim 13, or functionally active fragments thereof, in the manufacture of a medicament for the treatment of cancer.

15. A pharmaceutical formulation comprising the polypeptide of claim 13, and a pharmacologically acceptable carrier.

16. An antibody specific to the polypeptides of claim 13, or fragments, derivatives or homologues thereof.

17. An antibody according to claim 16, wherein said antibody is specific to the peptide sequence comprising the sequence of PYHESLAGASQPPYNPTYK or the sequence of YHETLAGGAAAPYPASQPPK.

18. A method for the diagnosis of cancer in a patient, said method comprising the detection of antibodies to an abnormal form of a protein, fragment or derivative thereof of the polypeptides of claim 13.

19. A method of treating diseases associated with abnormal cell proliferation comprising administering to a patient a therapeutic amount of the polypeptide of claim 13 in order to promote apoptosis in cells with abnormal proliferation.

20. A method according to claim 19, wherein said therapeutic amount of the polypeptide of claim 13 is administered to surface tumours.

21. A method of treating diseases associated with abnormal cell proliferation comprising administering to a patient a therapeutic amount of an agent which promotes apoptosis in cells with abnormal proliferation by increasing the expression and/or enhancing pro-apoptotic activity of the polypeptides of claim 13.

22. A method according to claim 21, wherein said treatment includes the application of an adenovirus containing the nucleotide sequences of any one of claims 1 to 4.

23. Use of the nucleotide sequences of FIGS. 2, 3, 13, 14, 15, 16, 17, 18 or 19, derivative or fragment thereof or species specific homologue thereof, or sequences complementary to said nucleotide sequences for determining a loss of expression of the p53-inducible gene.

24. Use of a nucleotide sequence according to claim 23, wherein said loss of expression is determined by northern blot analysis or RT-PCR.

25. Use of the sequence of FIGS. 2, 3, 13, 14, 15, 16, 17, 18 or 19, derivative or fragment thereof or species specific homologue thereof for isolating and identifying a promoter and/or regulatory sequence(s) associated with the any one of said sequences.

26. Use of a nucleotide sequence of FIGS. 2, 3, 13, 14, 15, 16, 17, 18 or 19, derivative or fragment thereof or species specific homologue thereof, in the manufacture of a medicament for the treatment of diseases associated with abnormal proliferation of cells.

27. Use of a nucleotide sequence according to claim 26, wherein said diseases are cancer or eczema.

28. Use of the nucleotide of FIGS. 2, 3, 13, 14, 15, 16, 17, 18 or 19, derivative or fragment thereof or species specific homologue thereof, and/or amino acids of FIGS. 4, 5, 20, 21, 22, 23 or 25, functionally active fragments, derivatives or homologues thereof, for the isolation and identification of agents, such as chemical compounds, which promote apoptosis.

29. Transgenic cells which comprise a polynucleotide fragment(s) comprising the nucleotide sequence of any one or more of the nucleotide sequences of FIGS. 2, 3, 13, 14, 15, 16, 17, 18 or 19, derivative or fragment thereof or species specific homologue thereof.

30. Transgenic cells according to claim 29, wherein said cells are mammalian cells.
